SEQUENCE LISTING (33 sequences)

SEQ ID NO: 1 - RNA, 20 nt, synthetic construct
  source 1..20: mol_type = other RNA
cgtagcccacggcgatgaag

SEQ ID NO: 2 - RNA, 20 nt, synthetic construct
  source 1..20: mol_type = other RNA
atttcttcacatccgtgtcc

SEQ ID NO: 3 - RNA, 20 nt, synthetic construct
  source 1..20: mol_type = other RNA
gtctcctggtcccaatactc

SEQ ID NO: 4 - RNA, 20 nt, synthetic construct
  source 1..20: mol_type = other RNA
gtagcccacggcgatgaagc

SEQ ID NO: 5 - RNA, 20 nt, synthetic construct
  source 1..20: mol_type = other RNA
tagcccacggcgatgaagcg

SEQ ID NO: 6 - RNA, 20 nt, synthetic construct
  source 1..20: mol_type = other RNA
gagggttcggggcgccatga

SEQ ID NO: 7 - RNA, 20 nt, synthetic construct
  source 1..20: mol_type = other RNA
ccggaacacacggaatgtga

SEQ ID NO: 8 - RNA, 20 nt, synthetic construct
  source 1..20: mol_type = other RNA
acagcgacgccgcgagccag

SEQ ID NO: 9 - RNA, 20 nt, synthetic construct
  source 1..20: mol_type = other RNA
ccactcacagattgaccgag

SEQ ID NO: 10 - RNA, 20 nt, synthetic construct
  source 1..20: mol_type = other RNA
cgacgccgcgagccagagga

SEQ ID NO: 11 - RNA, 20 nt, synthetic construct
  source 1..20: mol_type = other RNA
cagattgaccgagtggacct

SEQ ID NO: 12 - RNA, 20 nt, synthetic construct
  source 1..20: mol_type = other RNA
cagactgaccgagtggacct

SEQ ID NO: 13 - RNA, 20 nt, synthetic construct
  source 1..20: mol_type = other RNA
acagactgaccgagtggacc

SEQ ID NO: 14 - RNA, 20 nt, synthetic construct
  source 1..20: mol_type = other RNA
gccgggccgggacacggatg

SEQ ID NO: 15 - RNA, 20 nt, synthetic construct
  source 1..20: mol_type = other RNA
ccaggagacacggaatgtga

SEQ ID NO: 16 - RNA, 20 nt, synthetic construct
  source 1..20: mol_type = other RNA
ccagtcacagactgaccgag

SEQ ID NO: 17 - RNA, 20 nt, synthetic construct
  source 1..20: mol_type = other RNA
tcgacagcgacgccgcgagc

SEQ ID NO: 18 - RNA, 20 nt, synthetic construct
  source 1..20: mol_type = other RNA
gagatacatctacaaccggg

SEQ ID NO: 19 - RNA, 20 nt, synthetic construct
  source 1..20: mol_type = other RNA
tggagagatacatctacaac

SEQ ID NO: 20 - RNA, 20 nt, synthetic construct
  source 1..20: mol_type = other RNA
ggttgtagatgtatctctcc

SEQ ID NO: 21 - RNA, 20 nt, synthetic construct
  source 1..20: mol_type = other RNA
ggagagatacatctacaacc

SEQ ID NO: 22 - RNA, 20 nt, synthetic construct
  source 1..20: mol_type = other RNA
aggaatgctacgcgtttaat

SEQ ID NO: 23 - RNA, 20 nt, synthetic construct
  source 1..20: mol_type = other RNA
cagatacatctataaccgag

SEQ ID NO: 24 - RNA, 20 nt, synthetic construct
  source 1..20: mol_type = other RNA
cgagtactggaacagccaga

SEQ ID NO: 25 - RNA, 20 nt, synthetic construct
  source 1..20: mol_type = other RNA
cccgttggtgaagtagcaca

SEQ ID NO: 26 - RNA, 20 nt, synthetic construct
  source 1..20: mol_type = other RNA
gtgctacttcaccaacggga

SEQ ID NO: 27 - RNA, 20 nt, synthetic construct
  source 1..20: mol_type = other RNA
aggtcgtgcggagctccaac

SEQ ID NO: 28 - RNA, 20 nt, synthetic construct
  source 1..20: mol_type = other RNA
aggtgacagttgctacccga

SEQ ID NO: 29 - RNA, 20 nt, synthetic construct
  source 1..20: mol_type = other RNA
tcccgttggtgaagtagcac

SEQ ID NO: 30 - RNA, 20 nt, synthetic construct
  source 1..20: mol_type = other RNA
aactacgaggtggcgtaccg

SEQ ID NO: 31 - RNA, 20 nt, synthetic construct
  source 1..20: mol_type = other RNA
ctgttccagtactcggcaac

SEQ ID NO: 32 - RNA, 20 nt, synthetic construct
  source 1..20: mol_type = other RNA
actacgaggtggcgtaccgc

SEQ ID NO: 33 - RNA, 20 nt, synthetic construct
  source 1..20: mol_type = other RNA
cgaagcgcacgtactcctct

(19) United States
(12) Patent Application Publication
(10) Pub. No.: US 2026/0124241 A1
(43) Pub. Date: May 7, 2026

(54) MODIFIED HEMATOPOIETIC STEM CELLS AND PROGENIES THEREOF

(71) Applicant: Garuda Therapeutics, Inc., Cambridge, MA (US)

(72) Inventor: Dhvanit Shah, Chestnut Hill, MA (US)

(21) Appl. No.: 19/117,841

(22) PCT Filed: Oct. 5, 2023
(86) PCT No.: PCT/US2023/076091

§ 371 Date: Apr. 2, 2025

Related U.S. Application Data
(60) Provisional application No. 63/413,550, filed on Oct. 5, 2022.

(51) Int. Cl. A61K 35/17; C12N 5/00; C12N 5/0783; C12N 5/0789; C12N 9/22; C12N 15/11

(52) U.S. Cl. CPC A61K 35/17; C12N 5/0018; C12N 5/0636; C12N 5/0647; C12N 9/226; C12N 15/111; C12N 2310/20; C12N 2501/50; C12N 2506/45; C12N 2510/00

(57) ABSTRACT

The present disclosure in various aspects and embodiments relates to cell compositions (and methods for making or using the same) comprising hematopoietic stem cells (HSCs) and/or hematopoietic stem progenitor cells (HSPCs), where the HSCs and/or HSPCs have one or more endogenous genes modified in their expression, to thereby avoid or reduce targeting of these cells by targeted therapies. In an aspect, the disclosure provides such HSCs and/or HSPCs from gene-edited human induced pluripotent stem cells (iPSCs).

### CROSS-REFERENCE TO RELATED APPLICATIONS

This application claims the benefit of and priority to U.S. Provisional Patent Application No. 63/413,550 filed Oct. 5, 2022, the contents of which are hereby incorporated by reference in their entirety.

### SEQUENCE LISTING

The instant application contains a Sequence Listing which has been submitted in XML format via EFS-Web and is hereby incorporated by reference in its entirety. Said XML copy, created on Sep. 26, 2023, is named GRU-0010PC_Sequence_Listing.xml and is 30,045 bytes in size.

### BACKGROUND

An ideal antigen for targeted therapy is a disease-specific antigen, for example, a cancer-specific antigen. A cancer-specific antigen is an antigen that is exclusively expressed in malignant cells, thereby providing a unique target that will lead to maximal disease elimination with minimal off-target toxicity. However, such disease antigens are rare, as most antigens that are expressed by diseased or malignant cells are also found in normal tissues. For example, targeting the myeloid marker CD33 (e.g., using CAR-T cells) in acute myeloid leukemia (AML) results in toxicity from destruction of normal myeloid cells. To circumvent the loss or destruction of the normal cells, hematopoietic stem cells (HSCs) that have been genetically modified to disrupt expression of the targeted antigen provide a promising therapeutic approach to replenish normal cells that are lost or damaged due to the destruction of normal cells.

However, methods for making clinically relevant numbers of genetically-modified HSCs, and/or cell lineages therefrom, and having clinically advantageous phenotypes, remains a significant hurdle. There is a need to generate reliable, off-the-shelf, scalable HSCs (and progenies thereof) with one or more genetic modifications of cancer-associated antigens to treat diseases including but not limited to hematological cancers.

In various aspects and embodiments, the invention meets these objectives.

### SUMMARY OF THE DISCLOSURE

The present disclosure in various aspects and embodiments relates to cell compositions (and methods for making or using the same) comprising hematopoietic stem cells (HSCs) where the HSCs have one or more endogenous genes modified in their expression, to thereby avoid or reduce targeting of these cells by targeted therapies. In an aspect, the disclosure provides such HSCs from gene-edited human induced pluripotent stem cells (iPSCs).

In one aspect, the present disclosure provides a method for preparing a population of HSCs and/or hematopoietic stem progenitor cells (HSPCs) that are useful for replenishing hematopoietic cells in subjects undergoing a therapy that targets for destruction one or more hematopoietic lineages. The method comprises preparing a human iPSC population and modifying one or more endogenous genes in the iPSC population. Generally, the one or more endogenous genes comprise one or more cancer-associated antigens, and thus the present disclosure involves disrupting the expression of the cancer associated antigen(s) in the iPSC-derived HSCs or HSPCs. The method further comprises differentiating the iPSC population to a CD34+; population (e.g., recovered from dissociated embryoid bodies) and inducing endothelial-to-hematopoietic transition (ELT) of the CD34+ population to prepare a population comprising HSCs and/or HSPCs having reduced expression of the cancer-associated antigen. When these HSCs and/or HSPCs (or a cell population derived therefrom) are administered in connection with a cancer therapy that targets the cancer-associated antigen, targeting of the HSCs and/or HSPCs or their progeny can be reduced or avoided entirely.

In embodiments, the one or more endogenous genes are selected from CD33, CD19, CD7, CD123, and CD371, among others.

The HSCs and/or HSPCs can be derived from gene-edited iPSCs. In various embodiments, the iPSCs are prepared by reprogramming somatic cells, such as (without limitation) CD34+ cells isolated from peripheral blood. In embodiments, the iPSCs can be further gene edited to delete one or more of HLA-A, HLA-B, and HLA-C, and to delete one or more of HLA-DP, HLA-DQ, and HLA-DR. In certain embodiments, the iPSCs retain expression of at least one HLA Class I (e.g., of HLA-A, HLA-B, HLA-C, and ILA-E) and at least one HLA Class II complex. In certain embodiments, iPSCs are homozygous for at least one retained Class I and Class II loci. In some embodiments, the iPSCs are gene edited to be HLA-A^{neg}, homozygous for both HLA-B and HLA-C, and HLA-DPB1^{neg} and HLA-DQB1^{neg}. In some embodiments, the iPSCs are further homozygous for HLA-DRB1.

In some embodiments, the process of producing a population comprising HSCs and/or HSPCs can comprise generating CD34+-enriched cells from the differentiated pluripotent stem cells (e.g., EBs) and inducing endothelial-to-hematopoietic differentiation. HSCs comprising relatively high frequency of LT-HSCs can be generated from the cell populations using various stimuli or factors, including mechanical, biochemical, metabolic, and/or topographical stimuli.

Conventionally, hematopoietic stem cells or hematopoietic lineages are prepared by differentiation of iPSCs to embryoid bodies up to day 8 to harvest CD34+ cells. CD34 is commonly used as a marker of hemogenic endothelial cells, hematopoietic stem cells, and hematopoietic progenitor cells. In accordance with aspects and embodiments of this disclosure, it is discovered that inducing endothelial-to-hematopoietic transition (EHT) of a CD34+ cell population (e.g., for at least two days, but not more than 12 days), and which can be derived from iPSCs-embryoid bodies, can be used for the ex vivo generation of superior HSCs and hematopoietic lineages. The induction of endothelial-to-hematopoietic transition (EHT) can comprise increasing the expression or activity of dnmt3b, including pharmacologically with Piezol activation.

In various embodiments, CD34+ cells (e.g., the floater and/or adherent cells) are harvested from the culture undergoing endothelial-to-hematopoietic transition between Day 10 to Day 20 of iPSC differentiation. In some embodiments, non-adherent cells are collected.

In embodiments, the HSCs are further differentiated to a hematopoietic lineage for therapy. In embodiments, the hematopoietic lineage is selected from progenitor-T cells, T lymphocytes, and Natural Killer cells. Other lineages that can be produced and used for therapy include common myeloid progenitor (CMP) or common lymphoid progenitor (CLP) cells. CMPs give rise to progenies, such as, red blood cells/erythrocytes, platelets, mast cells, osteoclasts, granulocytes, monocyte-macrophages, and dendritic cells. CLPs give rise to progenies such as T-cells/T-lymphocytes, B-cells/B-lymphocytes, NK-cells/natural killer cells, and dendritic cells.

In some aspects and embodiments, the modified HSCs can be administered to replace normal cells that were destroyed by antibodies, T-cell therapy (e.g., CAR-T therapy), or NK cell therapy (e.g., CAR-NK). The HSCs and progenitors thereof help the bone marrow recover and make healthy cells, e.g., myeloid cells in AML treatment. HSC rescue allows more targeted therapy to be given to a patient so that more cancer cells are killed.

In other aspects, the present disclosure provides a cell population, or pharmaceutically acceptable composition thereof, produced by the method described herein. In various embodiments and as described herein, the HSC and/or HSPC population: is derived from iPSCs; has a genetically-disrupted expression of one or more endogenous genes that are cancer-associated antigens; proliferates in vivo but does not exhibit uncontrolled growth or tumor formation in vivo; and differentiates in vivo to reconstitute hematopoetic lineages.

In an aspect, the disclosure provides an HSC population that has a genetically-disrupted expression of one or more endogenous genes that are tumor-associated antigens; and wherein the iPSCs are: HLA-A^{neg}, homozygous for both HLA-B and HLA-C, HLA-DPB1^{neg}, HLA-DQB1^{neg}, and homozygous for HLA-DRB1. Such cells can be easily matched to a recipient for immune compatibility.

In various aspects and embodiments, the HSCs/HSPCs of the present disclosure may be used (e.g., in a method of treatment) to treat or ameliorate a disease or a disorder, such as treating a hematopoietic malignancy. Non-limiting examples of hematopoietic malignancies include, cytogenetically normal acute myeloid leukemia (CN-AML), acute myeloid leukemia (AML), acute lymphoid leukemia, chronic myelogenous leukemia, acute lymphoblastic leukemia or chronic lymphoblastic leukemia, chronic lymphoid leukemia, Hodgkin's lymphoma, non-Hodgkin's lymphoma, multiple myeloma or refractory or relapsing versions thereof. In some embodiments, the subject has, is, or will undergo a targeted therapy, for example, a therapy that targets hematopoietic cells (or lineage thereof).

In various embodiments, the modified HSCs and/or HSPCs of the disclosure (or cell lineage therefrom) is administered in connection with therapeutic applications of CAR-T therapy. Generally, the CAR-T cells target a cancer antigen that has reduced or eliminated expression in the modified HSCs. Exemplary CAR-T cells include CD33-specific CAR-T cells, CD7-specific CAR-T cells, CD8-specific CAR-T cells, CD19-specific CAR-T cells, CD20-specific CAR-T cells, CD22-specific CAR-T cells, CD123-specific CAR-T cells, CD125-specific CAR-T cells, CD133-specific CAR-T cells, and CD371-specific CAR-T cells.

In various embodiments, the HSCs and/or HSPCs or cell lineages derived therefrom (e.g., T cells or NK cells, including CAR-T cells or CAR-NK cells) are administered in connection with treatment of non-hematological malignancies, where the targeted antigen is expressed (even if at low levels) in normal hematopoietic cells or lineages.

Other aspects and embodiments of this disclosure will be apparent from the following detailed disclosure and working examples.

### DESCRIPTION OF THE FIGURES

FIG. 1 shows that ETV2 over-expression (OE) does not affect pluripotency. FIG. 1 shows FACS plots representative of transduction efficiency of iPSC with an adenoviral vector to overexpress ETV2 and GFP sequences. ETV2 overexpression does not affect the iPSC stemness as shown by the expression of the TRA-1-60 stemness marker.

FIG. 2 shows that ETV2 over-expression (OE) increases the yield of hemogenic endothelial cells. Representative flow cytometric analysis of hemogenic endothelial cells (described as CD235a-CD34+CD31+) and relative quantification demonstrates that ETV2-OE enhances the formation of hemogenic endothelial cells.

FIG. 3 shows that ETV2 over-expression (OE) enhances CD34+ cell formation during iPSC differentiation. Representative flow cytometric analysis of CD34+ cells and relative quantification demonstrates that ETV2-OE enhances the CD34+ cell formation.

FIG. 4A and FIG. 4B show that iPSC-derived HSCs that are derived with EHT of CD34+ cells (in this example with Piezol activation) undergo pro-T cell differentiation similar to bone marrow (BM)-HSCs. FIG. 4A is a FACS plot of differentiation efficiency to CD34+CD7+ pro T cells of Bone Marrow (BM) HSCs and iPSC-HSCs derived with EHT of CD34+ cells (with Piezol activation). FIG. 4B is a quantification of CD34+CD7+ cells (%) derived with (1) BM-HSCs and (2) iPSC-HSCs (Piezol Activation). FIG. 4B shows the average of three experiments.

FIG. 5A and FIG. 5B show that iPSC-derived HSCs generated with EHT of CD34+ cells (in this example with Piezol activation) undergo T cell differentiation and can be activated with CD3/CD28 beads similar to BM-HSCs. FIG. 5A is a FACS plot of activation efficiency (CD3+CD69+ expression) of T cells differentiated from BM-HSCs and iPSC-derived HSCs generated with EHT of CD34+ cells (in this example with Piezol activation). FIG. 5B is a quantification of CD3+CD69+ cells (%) derived with (1) BM-HSCs and (2) iPSC-HSCs (Piezol Activation). FIG. 5B shows the average of three experiments.

FIG. 6 shows that iPSC-derived HSCs generated with EHT of CD34+ cells (in this example with Piezol activation) can differentiate to functional T cells. IFNγ expression is a consequence of T cell activation after T cell receptor (TCR) stimulation via CD3/CD28 beads. IFNγ expression in T cells differentiated from iPSC-derived HSCs (EHT of CD34+ cells, generated upon Piezol activation), enhances ability to further differentiate to functional T cells. FIG. 6 shows the average of three experiments.

FIG. 7A-C show: generation of three CD33-KO iPSC clones (A), that CD33-KO does not affect the ability of cells to undergo the endothelial-to hematopoietic transition (B), and that CD33-KO does not affect the ability of cells to generate self-renewing HSCs (C).

FIG. 8A-C show: generation of three CCR5-knockout (KO) iPSC clones (A), that CCR5-KO does not affect the iPSC pluripotency (B), and that CCR5-KO does not affect the ability of cells to undergo the endothelial-to hematopoietic transition (C).

FIG. 9A and FIG. 9B show the phenotype analysis of triple knockout (HLA edited) cells performed by FACS and immunofluorescence. FIG. 9A shows the overall expression of HLA class-I molecules (HLA-A, HLA-B, and HLA-C) on the cell surface, where the HLA edited cells are positive for overall HLA class-I expression to a similar degree as wild-type cells (gHSCs). FIG. 9B shows cell expression of HLA-A via immunofluorescence, where HLA-A is not expressed in the HLA edited clone.

FIG. 10 shows that the HLA edited clones preserve their pluripotency (maintain trilineage differentiation), as illustrated by immunofluorescence, with ectoderm differentiation indicated by NESTIN-488 and PAX6-594 staining, mesoderm differentiation indicated by GATA-488 staining, and endoderm differentiation indicated by CXCR4-488 and FOX2A-594 staining.

FIG. 11 shows the immune compatibility of the HLA edited HSCs. HLA edited HSCs and control HSCs (WT, B2M KO, and HLA Class II null) were co-cultured with peripheral blood mononuclear cells (PBMCs) matching HLA-B and HLA-C, but with mismatched HLA-A. The PBMC-mediated cytotoxicity was measured by an annexin V staining assay.

FIG. 12 shows in vivo engrafting potential of HLA edited HSCs. Equal proportions of mCherry HLA edited HSCs and wild-type HSCs (gHSCs) were mixed for a competitive transplant into mice, where bone marrow (BM) and peripheral blood samples were evaluated by FACS to compare the relative amounts of each cell type present in the samples.

FIGS. 13A and 13B show that deletion of HLA-A does not impact Class I peptide presentation. FIG. 13A shows a schematic representation of immunopeptidome analysis. FIG. 13B shows results of the immunopeptidome analysis, which reveals that little difference exists in the numbers of peptides and representative proteins presented by class I molecules of WT and HLA-edited cells.

FIGS. 14A and 14B show that deletion of HLA-DP and DQ does not impact Class II peptide presentation. FIG. 14A shows immunopeptidome analysis scheme. FIG. 14B shows that despite the deletion of HLA-DP and DQ, the cells preserve their ability to present a broad spectrum of peptide through HLA Class II.

FIG. 15 is a schematic representation of in vivo testing of antigen-mediated immune response: Delayed Type Hypersensitivity Assay (DTH), sensitizing stage and elimination stage respectively.

FIGS. 16A and 16B show that HLA-edited HSCs reconstitute a functional immune system as demonstrated by DTH reaction in immune deficient mice. FIG. 16A shows a delayed-type hypersensitivity assay on transplanted mice were performed, which is an assay that involves the cross-talk of different types of immune cells. Mice were sensitized by subcutaneous injection of sheep Red blood cells (antigen). A functional immune system results in the swelling of the left paw that was measured with a micro caliper. As can be seen in FIG. 16A, the non-transplant mice did not show any left paw swelling as they are immunodeficient. Conversely, the mice transplanted with Cord Blood CD34+ cells show tissue swelling and doubled the diameter of their left paw. FIG. 16B is a graphical evaluation of the results shown in FIG. 16A.

FIG. 17 shows the HSC differentiation potential into T cell subtypes. After a 35-day differentiation period pro-T cells were evaluated by cell sorting for the presence of CD4+, CD8+, and AB+ T cell populations. FIG. 17 compares the differentiation potential of bone marrow-derived CD34+ cells, embryoid body CD34+ cells, and HSCs prepared according to the present disclosure (e.g., using Piezol activation) (“gHSCs”).

FIG. 18 shows the degree of T-cell mediated cytotoxicity measured from a co-culture of HSC-derived T cells with CD19+ lymphoma cells in the presence of an anti-CD3/CD-19 bispecific antibody. T cells prepared from HSCs according to the present disclosure (“gHSC”) demonstrate a high level of cytotoxicity against the target cells.

FIG. 19 shows that HSC-derived T cells (pro-T cells) can be transduced with high efficiency. Pro-T cells underwent lentiviral (LV) transduction with an anti-CD-19 chimeric antigen receptor (CAR) transgene (left), where the efficiency of LV transduction was measured by cell sorting based on anti-CD19 scFv staining (right). Results indicate that HSC-derived T cells achieved approx. 85% transduction efficiency.

FIG. 20 shows that the HSC-derived pro T cells can effectively mature into CD4+/CD8+ T cells via CAR transduction.

FIG. 21 shows the ability of anti-CD19 CAR-transduced HSC-derived T cells (CAR pro-T cells) to function via receptor-mediated cytotoxicity. Luc+ NALM6 leukemia cells were co-cultured with CAR pro-T cells and cell-mediated cytotoxicity was measured by luciferase assay.

FIG. 22 shows the ability of the HSCs prepared according to this disclosure (with Piezol activation) to develop into pro-T cells as measured by their CD34-CD7+ markers.

FIGS. 23A and 23B demonstrates increased expression of T cell-specific transcription factors and Thymus engrafting molecules with the pro-T cells derived from HSCs according to the instant disclosure. FIG. 23A shows TCF7 mRNA expression and FIG. 23B shows CCR7 mRNA expression.

FIGS. 24A and 24B shows that HSC-derived Pro-T Cells engraft and differentiate in thymus. FIG. 24A illustrates the engraftment and analysis procedure. FIG. 24B shows FACS analysis of CD3 cell population of cells gated on CD45+ cell population, which shows the superior engraftment and differentiation potential of the HSC-derived Pro-T Cells in the thymus.

FIG. 25 shows HSC-derived T cells can be activated in vitro. Top panel shows FACS analysis of activated T cells from different sources, including from HSCs prepared according to the present disclosure. T cells of the present disclosure demonstrate comparable or superior activation as measured by increased CD107 expression. The lower panel shows Dynabeads activation, where activated T cells express inflammatory cytokines. HSC-derived T cells express higher levels of inflammatory cytokines as exemplified by TNF-alpha and interferon gamma expression levels.

FIG. 26 shows that CCR5-knocked out HSCs can comparably differentiate into pro-T cells, compared to their wild type (gHSC) counterpart HSC (CCR5 retained).

FIG. 27 shows CCR5-knocked out HSCs can comparably differentiate into double positive (CD4+CD8+) T cells when compared to their wild type counterpart HSCs (CCR5 retained).

The term “gHSC” is used herein to refer to the iPSC-derived hematopoietic stem cells of the present disclosure.

The terms “wild type” (WT), “unedited”, “non-HLA-edited” are used interchangeability herein to refer to the non-gene edited cells of the present disclosure.

EB34+ cells refer to Embryonic body derived CD34+ cells. These comprise hemogenic endothelial cells.

### DETAILED DESCRIPTION

The present disclosure in various aspects and embodiments relates to cell compositions (and methods for making or using the same) comprising hematopoietic stem cells (HSCs), where the HSCs have one or more endogenous genes modified in their expression, to thereby avoid or reduce targeting of these cells by targeted therapies. In an aspect, the disclosure provides such HSCs from gene-edited human induced pluripotent stem cells (iPSCs).

In one aspect, the present disclosure provides a method for preparing a population of HSCs and/or hematopoietic stem progenitor cells (ISPCs) that are useful for replenishing hematopoietic cells in subjects undergoing a therapy that targets for destruction one or more hematopoietic lineages. The method comprises preparing a human iPSC population and modifying one or more endogenous genes in the iPSC population. Generally, the one or more endogenous genes comprise one or more cancer-associated antigens, and thus the present disclosure involves disrupting the expression of the cancer associated antigen(s) in the iPSC-derived HSCs. The method further comprises differentiating the iPSC population to a CD34+ population (e.g., recovered from dissociated embryoid bodies) and inducing endothelial-to-hematopoetic transition (ET-IT) of the CD34+ population to prepare a population comprising HSCs and/or HSPCs having reduced expression of the cancer-associated antigen. EHT can be induced for example for at least 2 days, and up to 12 days. When these HSCs and/or HSPCs (or a cell population derived therefrom) are administered in connection with a cancer therapy that targets the cancer-associated antigen, targeting of the HSCs and/or HSPCs or their progeny can be reduced or avoided entirely.

One of the greatest complexities in treating diseases such as cancer by targeting an antigen associated with the diseased cells, is that most antigens are also present in normal cells. For example, if CD33 is targeted by a T cell therapy (e.g., CAR-T cell), for example as a treatment for acute myeloid leukemia (AML), CD33 will also be present on normal myeloid cells, leading to the destruction of normal myeloid cells. Thus, administration of the HSCs described herein (or a progeny thereof) to reconstitute hematopoietic lineages can avoid this effect by reducing or eliminating expression of CD33. This same approach can be applied to various cancer associated antigens that are useful for targeting with immune therapies.

As used herein, a “cancer associated antigen” or “tumor associated antigen” is an antigen that is expressed in cancer or tumor cells. In some embodiments, the cancer associated antigen is expressed on the surface of cancer or tumor cells. In some embodiments, the antigen is expressed at higher levels in the cancer cells as compared to non-malignant cells (i.e., normal cells). In other embodiments, the antigen is expressed in a cell lineage, but at comparable levels in malignant and normal cells. Various cancer-associated antigens are described herein. In some embodiments, the cancer-associated antigen is expressed or overexpressed in a hematological cancer. For example, the cancer-associated antigen may be expressed or overexpressed in one or more of cytogenetically normal acute myeloid leukemia (CN-AML), acute myeloid leukemia (AML), acute lymphoid leukemia, chronic myelogenous leukemia, acute lymphoblastic leukemia, chronic lymphoblastic leukemia, chronic lymphoid leukemia, Hodgkin's lymphoma, non-Hodgkin's lymphoma, and multiple myeloma. In embodiments, the cancer-associated antigen is predominantly expressed on immune cells (in comparison to other tissues). In some embodiments, the cancer-associated antigen is predominantly expressed on myeloid cells. That is, the cancer associated antigen is expressed on myeloid cells at higher levels than in other cells and tissues. In some embodiments, the cancer-associated antigen is predominantly expressed on thymocytes. In some embodiments, the cancer-associated antigen is predominantly expressed in T cells or in B cells. In some embodiments, the cancer-associated antigen is expressed in immune cells at all stages of cell differentiation. In some embodiments, at least one endogenous gene (comprising a cancer associated antigen) that is genetically modified is selected from CD33, CD19, CD7, CD123, and CD371.

In various embodiments, the HSCs and/or HSPCs can have a reduced expression of the endogenous gene, or in some embodiments expression of the endogenous gene is eliminated. For example, in accordance with embodiments, at least about 50%, or at least about 75%, or at least about 90%, or about 100% of the HSCs (or their progeny) do not express the endogenous gene. Alternatively, at least about 50%, or at least about 75%, or at least about 90%, or about 100% of the HSCs and/or HSPCs (or their progeny) express a reduced expression level of the endogenous gene. In some embodiments, all or portions of the endogenous gene are deleted to eliminate its expression. Alternatively, cis or trans expression control factors can be genetically manipulated to reduce, but not eliminate, expression. For example, the expression can be reduced by at least 50% as compared to HSCs (or their progeny) that do not comprise gene editing of the cis or trans expression control factors. In some embodiments, the HSCs and/or HSPCs have a deletion or inactivation of only one copy of the endogenous gene (i.e., thereby eliminating expression of one copy of the endogenous gene), leading to reduced expression. Alternatively, the HSCs and/or HSPCs have a deletion or inactivation of both copies of the endogenous gene (thereby eliminating expression entirely).

In embodiments, the one or more endogenous genes includes CD33. In such embodiments, the HSCs and/or HSPCs with reduced or eliminated expression of CD33 are suitable for myeloid differentiation. For example, the HSCs and/or HSPCs differentiate into myeloid and lymphoid progenitors as well as mature myeloid and lymphoid cells.

In embodiments, the one or more endogenous genes includes CD19. In such embodiments, the HSCs with reduced or eliminated expression of CD19 are suitable for B lymphocyte differentiation. In embodiments, the HSCs and/or HSPCs differentiate into myeloid and lymphoid progenitors as well as mature myeloid and lymphoid cells.

In embodiments, the one or more endogenous genes includes CD7. In such embodiments, the HSCs and/or HSPCs with reduced or eliminated expression of CD7 are suitable for T cell progenitor differentiation. In embodiments, the HSCs and/or HSPCs differentiate into myeloid and lymphoid progenitors as well as mature myeloid and lymphoid cells.

In embodiments, the one or more endogenous genes includes CD123. In such embodiments, the HSCs with reduced or eliminated expression of CD123 are suitable for myeloid differentiation. In embodiments, the HSCs and/or HSPCs differentiate into myeloid and lymphoid progenitors as well as mature myeloid and lymphoid cells.

In embodiments, the one or more endogenous genes includes CD371. In such embodiments, the HSCs with reduced or eliminated expression of CD371 are suitable for myeloid differentiation. In embodiments, the HSCs and/or HSPCs differentiate into myeloid and lymphoid progenitors as well as mature myeloid and lymphoid cells therefrom.

In various embodiments, one, two, three, or four endogenous genes comprising tumor associated antigens are genetically modified to reduce or eliminate expression, and where one or two of such genes are selected from CD33, CD19, CD7, CD123, and CD371. Other exemplary tumor associated antigens are selected from CD1b, CD1c, CD1d, CD1e, CD2, CD3, CD3d, CD3e, CD3g, CD4, CD5, CD6, CD8a, CD8b, CD9, CD10, CD11a, CD11b, CD11c, CD11d, CDwl2, CD13, CD14, CD15, CD15u, CD15s, CD15su, CD16, CD16b, CD17, CD18, CD20, CD21, CD22, CD23, CD24, CD25, CD26, CD27, CD28, CD29, CD30, CD31, CD32, CD34, CD35, CD36, CD37, CD38, CD39, CD40, CD41, CD42a, CD42b, CD42c, CD42d, CD43, CD44, CD45, CD45RA, CD45RB, CD45RC, CD45RO, CD46, CD47, CD48, CD49a, CD49b, CD49c, CD49d, CD49e, CD49f, CD50, CD51, CD52, CD53, CD54, CD55, CD56, CD57, CD58, CD59, CD60a, CD60b, CD60c, CD61, CD62E, CD62L, CD62P, CD63, CD64, CD65, CD65s, CD66a, CD66b, CD66c, CD66d, CD66e, CD66f, CD68, CD69, CD70, CD71, CD72, CD73, CD74, CD75, CD75s, CD77, CD79a, CD79b, CD80, CD81, CD82, CD83, CD84, CD85a, CD85d, CD85j, CD85k, CD86, CD87, CD88, CD89, CD90, CD91, CD92, CD93, CD94, CD95, CD96, CD97, CD98, CD99, CD99R, CD100, CD101, CD102, CD103, CD104, CD105, CD106, CD107a, CD107b, CD108, CD109, CD110, CD111, CD112, CD113, CD114, CD115, CD116, CD117, CD118, CD119, CD120a, CD120b, CD121a, CD121b, CD122, CD124, CD125, CD126, CD127, CD129, CD130, CD131, CD132, CD133, CD134, CD135, CD136, CD137, CD138, CD139, CD140a, CD140b, CD141, CD142, CD143, CD144, CDw145, CD146, CD147, CD148, CDwl49, CD150, CD151, CD152, CD153, CD154, CD155, CD156a, CD156b, CD156c, CD157, CD158e, CD158i, CD158k, CD159a, CD159c, CD160, CD161, CD162, CD163, CD164, CD165, CD166, CD167a, CD167b, CD168, CD169, CD170, CD171, CD172a, CD172b, CD172g, CD173, CD174, CD175, CD175s, CD176, CD177, CD178, CD179a, CD179b, CD180, CD181, CD182, CD183, CD184 (CXCR4), CD185, CD186, CD191, CD192, CD193, CD194, CD195, CD196, CD197, CDw198, CD199, CD200, CD201, CD202b, CD203c, CD204, CD205, CD206, CD207, CD208, CD209, CD210, CDw210b, CD212, CD213a1, CD213a2, CD215, CD217a, CD218a, CD218b, CD220, CD221, CD222, CD223, CD224, CD225, CD226, CD227, CD228, CD229, CD230, CD231, CD232, CD233, CD234, CD235a, CD235b, CD236, CD236R, CD238, CD239, CD240CE, CD240DCE, CD240D, CD241, CD242, CD243, CD244, CD245, CD246, CD247, CD248, CD249, CD252, CD253, CD254, CD256, CD266, CD267, CD268, CD269, CD270, CD271, CD272, CD273, CD274, CD275, CD276, CD277, CD278, CD279, CD280, CD281, CD282, CD283, CD284, CD286, CD289, CD290, CD292, CDw293, CD294, CD295, CD296, CD297, CD298, CD299, CD300a, CD300c, CD300e, CD301, CD302, CD303, CD304, CD305, CD306, CD307a, CD307b, CD307c, CD307d, CD307e, CD308, CD309, CD312, CD314, CD315, CD316, CD317, CD318, CD319, CD320, CD321, CD322, CD324, CD325, CD326, CD327, CD328, CD329, CD331, CD332, CD333, CD334, CD335, CD336, CD337, CD338, CD339, CD340, CD344, CD349, CD350, CD351, CD352, CD353, CD354, CD355, CD357, CD358, CD360, CD361, CD362, CD363, CD364, CD365, CD366, CD367, CD368, CD369, and CD370.

In some embodiments, at least one endogenous gene comprises a growth factor receptor, such as one or more selected from ErbB1, ErbB2, ErbB3, or ErbB4, IGF1R, IGF2R, TβR I-II, VEGFR1, VEGFR2, VEGFR3, PDGFR (α/β), and FGFR1, 2, 3 or 4.

The HSCs and/or HSPCs are derived from gene-edited iPSCs. In various embodiments, the iPSCs are prepared by reprogramming somatic cells. The term “induced pluripotent stem cell” or “iPSC” refers to cells derived from somatic cells, such as skin or blood cells that have been reprogrammed back into an embryonic-like pluripotent state. In some embodiments, iPSCs are generated from somatic cells such as (but not limited to) fibroblasts or PBMCs (or cells isolated therefrom). In some embodiments, the iPSCs are derived from lymphocytes (e.g., T-cells, B-cells, NK-cells, etc.), cord blood cells (including from CD3+ or CD8+ cells from cord blood), PBMCs, CD34+ cells, or other human primary tissues. In some embodiments, iPSCs are derived from CD34+ cells isolated from peripheral blood. In various embodiments, the iPSCs are autologous or allogenic (e.g., HLA-matched at one or more loci) with respect to a recipient (a subject in need of treatment as described herein). In various embodiments, the iPSCs can be gene edited to assist in HLA matching (such as deletion of one or more HLA Class I and/or Class II alleles or their master regulators, including but not limited beta-2-microglobulin (B2M), CIITA, etc.). For example, iPSCs can be gene edited to delete one or more of HLA-A, HLA-B, and HLA-C, and to delete one or more of HLA-DP, HLA-DQ, and HLA-DR. In certain embodiments, the iPSCs retain expression of at least one HLA Class I (e.g., of HLA-A, HLA-B, HLA-C, and HLA-E) and at least one HLA Class II complex. In certain embodiments, iPSCs are homozygous for at least one retained Class I and Class II loci.

In various embodiments, HSC and/or HSPC populations are derived from iPSCs which are gene edited to be one of: (i) HLA-A-B+C+DP−DR+DQ+, (ii) HLA-A-B+C+DP+DR+DQ−, (iii) HLA-A-B+C+DP−DR+DQ−, (iv) HLA-A-B-C+DP−DR+DQ+; (v) HLA-A-B-C+DP+DR+DQ−, (vi) HLA-A-B-C+DP−DR+DQ−. For retained HLA (for example HLA-B, HLA-C, and HLA-DR), cells can be homozygous or retain only a single copy of the gene. For example, the modified cells are identified at least as (a) HLA-C+ and ILA-DR+, and optionally identified as one or more of (b) HLA-B−, (c) HLA-DP−, and (d) HLA-DQ−. In exemplary embodiments, the modified cells are HLA-B+, HLA-DP−, and HLA-DQ−.

In some embodiments, HSC and/or HSPC populations are derived from iPSCs that are gene edited to be HLA-A^{neg}, homozygous for both HLA-B and HLA-C, and HLA-DPBI1^{neg} and HLA-DQB1^{nee}. In some embodiments, the iPSCs are further homozygous for HLA-DRB1.

As used herein, the term “neg,” (−), or “negative,” with respect to a particular HLA Class I or Class II molecule indicates that both copies of the gene have been disrupted in the cell line or population, and thus the cell line or population does not display significant functional expression of the gene. Such cells can be generated by full or partial gene deletions, or alternatively with other technologies such as siRNA. As used herein, the term “delete” in the context of a genetic modification of a target gene (i.e., gene edit) refers to abrogation of functional expression of the corresponding gene product (i.e., the corresponding polypeptide). Such gene edits include full or partial gene deletions or disruptions, or deletions of critical cis-acting expression control sequences.

In certain embodiments, the disruption of the one or more endogenous genes (such as the endogenous gene encoding a cancer associated antigen), as well as one or more HLA Class I and/or Class II genes in some embodiments, or genes governing HLA expression or presentation capacity, is generated by introducing a Cas9 endonuclease or a nucleic acid encoding a Cas9 endonuclease to the iPSCs, as well as a nucleic acid molecule encoding a guide RNA (gRNA) directing the mutation or deletion of nucleotide sequences of the endogenous gene by the Cas9 endonuclease.

In some embodiments, the iPSCs are gene edited using gRNAs that are 16, 17, 18, 19, 20, 21, 22, 23, 24, 25, 26 or more nucleotides in length. In some embodiments, the gRNAs comprise a modification at or near the 5′ end (e.g., within 1-10, 1-5, or 1-2 nucleotides of the 5′ end) and/or a modification at or near the 3′ end (e.g., within 1-10, 1-5, or 1-2 nucleotides of the 3′ end). In some embodiments, the modified gRNAs exhibit increased resistance to nucleases. In some embodiments, a gRNA comprises two separate RNA molecules (i.e., a “dual gRNA”). A dual gRNA comprises two separate RNA molecules: a “crispr RNA” (or “crRNA”) and a “tracr RNA” and is well known to one of skill in the art.

Generally, various gene editing technologies are known, which can be applied according to various embodiments of this disclosure. Gene editing technologies include but are not limited to zinc fingers (ZFs), transcription activator-like effectors (TALEs), etc. Fusion proteins containing one or more of these DNA-binding domains and the cleavage domain of Fokl endonuclease can be used to create a double-strand break in a desired region of DNA in a cell (See, e.g., US Patent Appl. Pub. No. US 2012/0064620, US Patent Appl. Pub. No. US 2011/0239315, U.S. Pat. No. 8,470,973, US Patent Appl. Pub. No. US 2013/0217119, U.S. Pat. No. 8,420,782, US Patent Appl. Pub. No. US 2011/0301073, US Patent Appl. Pub. No. US 2011/0145940, U.S. Pat. Nos. 8,450,471, 8,440,431, 8,440,432, and US Patent Appl. Pub. No. 2013/0122581, the contents of all of which are hereby incorporated by reference). In some embodiments, gene editing is conducted using CRISPR associated Cas system (e.g., CRISPR-Cas9), as known in the art. See, for example, U.S. Pat. Nos. 8,697,359, 8,906,616, and 8,999,641, each of which is hereby incorporated by reference in its entirety. In various embodiments, the gene editing employs a Type II Cas endonuclease (such as Cas9) or employs a Type V Cas endonuclease (such as Cas12a). Type II and Type V Cas endonucleases are guide RNA directed. Design of gRNAs to guide the desired gene edit (while limiting or avoiding off target edits) is known in the art. See, for example, Mohr S E, et al., CRISPR guide RNA design for research applications, FEBS J. 2016 September; 283(17): 3232-3238. In still other embodiments, non-canonical Type II or Type V Cas endonucleases having homology (albeit low primary sequence homology) to S. pyogenes Cas9 or Prevotella and Francisellal (Cpf1 or Cas12a) can be employed. Numerous such non-canonical Cas endonucleases are known in the art. Nidhi S, et al. Novel CRISPR-Cas Systems: An Updated Review of the Current Achievements, Applications, and Future Research Perspectives, Int J Mol Sci. 2021 April; 22(7): 3327. In still other embodiments, the gene editing employs base editing or prime editing to incorporate mutations without instituting double strand breaks. See, for example, Antoniou P, et al., Base and Prime Editing Technologies for Blood Disorders, Front. Genome Ed., 28 Jan. 2021; Matsuokas IG, Prime Editing: Genome Editing for Rare Genetic Diseases Without Double-Strand Breaks or Donor DNA, Front. Genet., 9 Jun. 2020. Various other gene editing processes are known, including use of dead Cas (dCas) systems (e.g., Cas fusion proteins) to target DNA modifying enzymes to desired targets using the dCas as a guide RNA-directed system. Brezgin S, Dead Cas Systems: Types, Principles, and Applications, Int J Mol Sci. 2019 December; 20(23): 6041.

Base editors that can install precise genomic alterations without creating double-strand DNA breaks can also be used in gene editing (e.g., designing gene therapy vectors) in the cells (e.g., iPSCs). Base editors essentially comprise a catalytically disabled nuclease, such as Cas9 nickase (nCas9), which is incapable of making DSBs and is fused to a nucleobase deaminase enzyme and, in some cases, a DNA glycosylase inhibitor. Currently, there are 2 major categories of base editors, cytidine base editors (CBEs) and adenine base editors (ABEs), which catalyze C>T and A>G transitions. Base editors can be delivered, for example, via HDAd5/35++ vectors to efficiently edit promoters and enhancers to active or inactivate a gene. Exemplary methods are described in U.S. Pat. Nos. 9,840,699; 10,167,457; 10,113,163; 11,306,324; 11,268,082; 11,319,532; and 11,155,803. Also contemplated are prime editors that comprise a reverse transcriptase conjugated to (e.g., fused with) a Cas endonuclease and a polynucleotide useful as a DNA synthesis template conjugated to (e.g., fused with) a guide RNA, as described in WO 2020/191153.

Exemplary vectors that can be used for the genome editing applications include, but are not limited to, plasmids, retroviral vectors, lentiviral vectors, adenovirus vectors (e.g., Ad5/35, Ad5, Ad26, Ad34, Ad35, Ad48, parvovirus (e.g., adeno-associated virus (AAV) vectors, herpes simplex virus vectors, baculoviral vectors, coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including herpes virus (e.g., Herpes Simplex virus types 1 and 2, Epstein-Barr virus, cytomegalovirus), and poxvirus (e.g., canarypox, vaccinia or modified vaccinia virus. The vector comprising the nucleic acid molecule of interest may be delivered to the cell (e.g., iPS cells, endothelial cells, hemogenic endothelial cells, HSCs (ST-HSCs or LT-HSCs) via any method known in the art, including but not limited to transduction, transfection, infection, and electroporation. Any of these vectors may include transposable element (such as a piggyback transposon or sleeping beauty transposon). Transposons insert specific sequences of DNA into genomes of vertebrate animals. The gene of interest can be integrated into the genome of a mammalian cell by transposase-catalyzed cleavage of similar excision sites that exist within the nuclear genome of the cell.

For increased efficiency, in some embodiments, the Cas and the gRNA can be combined before being delivered into the cells. The Cas-gRNA complex is known as a ribonucleoprotein (RNP). A number of methods have been developed for direct delivery of RNPs to cells. For example, RNP can be delivered into cells in culture by lipofection or electroporation. Electroporation using a nucleofection protocol can be employed, and this procedure allows the RNP to enter the nucleus of cells quickly, so it can immediately start cutting the genome. See, for example, Zhang S, Shen J, Li D, Cheng Y. Strategies in the delivery of Cas9 ribonucleoprotein for CRISPR/Cas9 genome editing. Theranostics. 2021 Jan. 1; 11(2):614-648, hereby incorporated by reference in its entirety. In some embodiments, Cas9 and gRNA are electroporated as RNP into the donor iPSCs and/or HSCs.

Generally, a protospacer adjacent motif (PAM) is required for a Cas nuclease to cut and is generally found 3-4 nucleotides downstream from the cut site. The PAM is a short DNA sequence (usually 2-6 base pairs in length) that follows the DNA region targeted for cleavage by the CRISPR system, such as CRISPR-Cas9. In some embodiments, the PAM sequences, sgRNAs, or base editing tools targeting haplotypes or polymorphs of HLA loci does not include four Gs, four Cs, GC repeats, or combinations thereof.

In some embodiments, a CRISPR/Cas9 system specific to a unique HLA haplotype can be developed by designing singular gRNAs targeting each of the donor-specific HLA-A, HLA-DPB1, and HLA-DQB1 genes (for example), using the gRNAs as described herein. To perform genetic knockout, the gRNA targets the Cas9 protein to the appropriate site to edit. Next, the Cas9 protein can perform a double strand break (DSB), where the DNA repairs through a non-homologous end joining (NHEJ) mechanism which generates indels resulting in a frameshift mutation and terminates the resulting protein's function. However, off-target genetic modifications can occur and alter the function of otherwise intact genes. For example, the Cas9 endonuclease can create DSBs at undesired off-target locations, even in the presence of some degree of mismatch. This off-target activity can create genome instability events, such as point mutations and genomic structural variations. In various embodiments, a sgRNA targeting HLA-A can target a region of chromosome 6 defined as 29942532-29942626. In various embodiments, a sgRNA targeting HLA-DQB1 can target a region of chromosome 6 defined as 32665067-32664798. In various embodiments, a sgRNA targeting HLA-DPB1 can target a region of chromosome 6 defined as 33080672-33080935.

gRNAs can be used to develop clonal iPSCs. Such iPSC lines can be evaluated for (i) ON-target edits, (ii) OFF-target edits, and (iii) Translocation edits, for example using sequencing, as described herein. Specifically, such assays can be performed by multiplex PCR with primers designed to target and enrich regions of interest followed by next-generation sequencing (e.g., Amplicon sequencing, AMP-seq). The ON-target panel and the translocation panel can amplify the intended edited region, allowing for selection of iPSC clones with the expected edits which are free from chromosomal translocation arising from unintended DSB cut-site fusion. The OFF-target panel can enrich any potential off-target regions identified via sequencing and allows for selection of iPSC clones with negligible off-target mutations. Together, these assays enable a screen of the iPSC clones to select the clones with the desired edits, while excluding potential CRISPR/Cas9-related genome integrity issues.

In some embodiments, to further ensure the genomic stability and integrity of reprogrammed and edited iPSCs, genetic and genomic assays can be performed to select for clones which, for example, did not undergo translocation and mutation events, and that did not integrate the episomal vectors. For example, whole-genome sequencing (WGS) is performed on CD34+ cells and on iPSC clones after reprogramming, where the genomes are compared for differences arising from editing. These analyses provide an assessment of which iPSC clone genomes differ from the CD34+ starting material, enabling informed selection iPSC clones which did not accrue mutations during the reprogramming.

In some embodiments, karyotyping analyses using systems such as KARYOSTAT assays is used to select iPSC clones which did not accrue indels and translocation during the reprogramming, for example as described in Ramme A P, et al, “Supporting dataset of two integration-free induced pluripotent stem cell lines from related human donors,” Data Brief. 2021 May 15; 37:107140, hereby incorporated by reference in its entirety. KARYOSTAT assays allow for visualization of chromosome aberrations with a resolution similar to G-banding karyotyping. The size of structural aberration that can be detected is >2 Mb for chromosomal gains and >1 Mb for chromosomal losses. The KARYOSTAT array is functionalized for balanced whole-genome coverage with a low-resolution DNA copy number analysis, where the assay covers all 36,000 RefSeq genes, including 14,000 OMIM targets. The assay enables the detection of aneuploidies, submicroscopic aberrations, and mosaic events.

In some embodiments, Array Comparative Genomic Hybridization (aCGH) analyses is used to select iPSC clones which did not accrue copy number aberrations (CNA) during reprogramming, for example as described in Wiesner et al. “Molecular Techniques,” Editor(s): Klaus J. Busam, Pedram Gerami, Richard A. Scolyer, “Pathology of Melanocytic Tumors,” Elsevier, 2019, pp. 364-373, ISBN 9780323374576; and Hussein S M, et al. “Copy number variation and selection during reprogramming to pluripotency,” Nature. 2011 Mar. 3; 471(7336):58-62, hereby incorporated by reference in its entirety. aCGH is a technique that analyzes the entire genome for CNA by comparing the sample DNA to reference DNA.

In some embodiments, targeted heme malignancy NGS panel analyses is used to select iPSC clones which did not accrue hematologic malignancy mutations during reprogramming. For example, targeted heme malignancy NGS panels can focus on myeloid leukemia, lymphoma, and/or other hematologic malignancy-associated genes to generate a smaller, more manageable data set than broader methods. Targeted heme malignancy NGS panel analysis includes the use of highly multiplexed PCR to amplify regions associated with hematologic malignancies followed by next-generation sequencing.

In some embodiments, Droplet Digital PCR (ddPCR) is used to select iPSC clones which did not integrate episomal vectors and that have been passaged enough for episomal vector clearance. As discussed herein, iPSC reprogramming of CD34+ cells can be achieved by delivering episomal vectors encoding reprogramming factors. However, episomal vectors can, albeit rarely, randomly integrate into the cellular genome, which could disrupt developmental processes, homeostasis, etc. Therefore, ddPCR methods can be used to detect residual episomal vector in the iPSC cultures and enable selection of iPSC clones which did not integrate episomal vectors.

In some embodiments, after assessing that the selected clones are free from genomic aberrations related to editing, the clones can be additionally tested for spontaneous mutations that might arise during expansion. For example, mutations affecting hematologic malignancy genes, indel, translocations, number aberrations, e.g., as described for the pre-edited reprogrammed clones. Analyses for spontaneous mutations can include whole-genome sequencing (WGS), KARYOSTAT analysis, Array Comparative Genomic Hybridization (aCGH) analysis, targeted heme malignancy NGS panel AMP-Seq analysis, and/or Droplet Digital PCR (ddPCR).

Somatic cells may be reprogrammed by expression of reprogramming factors selected from Sox2, Oct3/4, c-Myc, Nanog, Lin28, and klf4. In some embodiments, the reprogramming factors are Sox2, Oct3/4, c-Myc, Nanog, Lin28, and klf4. In some embodiments, the reprogramming factors are Sox2, Oct3/4, c-Myc, and klf4. Methods for preparing iPSCs are described, for example, in U.S. Pat. Nos. 10,676,165; 9,580,689; and 9,376,664, which are hereby incorporated by reference in their entireties. In various embodiments, reprogramming factors are expressed using well known viral vector systems, such as lentiviral, Sendai, or measles viral systems. Alternatively, reprogramming factors can be expressed by introducing mRNA(s) encoding the reprogramming factors into the somatic cells. Further still, iPSCs may be created by introducing a non-integrating episomal plasmid expressing the reprogramming factors, i.e., for the creation of transgene-free and virus-free iPSCs. Known episomal plasmids can be employed with limited replication capabilities and which are therefore lost over several cell generations.

In some embodiments, iPSCs are derived from T cells, for example, with a known or unknown TCR specificity. In some embodiments, the T cells bear TCRs with specificity for cancer or tumor associated antigens, and which in some embodiments comprise a cancer or tumor associated antigen that is deleted or reduced in expression in the iPSCs. For example, the iPSCs can be prepared from CD3⁺ cells or in some embodiments T lymphocytes (e.g., CTLs) (T-iPSCs). For example, T lymphocytes can be isolated with a desired antigen specificity (using for example, cell sorting with HLA-peptide ligands), and reprogrammed to T-iPSCs. These T-iPSCS are then re-differentiated into HSCs, or optionally progenitor T cells, or T cell lineages. When T-iPSCs are produced from antigen-specific T cells, T-iPSCs inherit the rearranged T cell receptor (TCR) genes. In these embodiments, CTLs that are derived from the HSCs demonstrate the same antigen specificity as the original antigen-specific T cells.

In some embodiments, the iPSCs can be further engineered by inserting at least one sequence encoding a transgene operatively linked to an endogenous or exogenous promoter, wherein the transgene is inserted within a genomic safe harbor locus. A genomic safe harbor (GSH) locus refers to a genetic locus that accommodates the insertion of exogenous DNA with either constitutive or conditional expression activity without significantly affecting the viability of somatic cells, progenitor cells, or germ line cells and ontogeny. Well-known safe harbor locus include the AAVS1 adeno-associated virus insertion site on chromosome 19, the human homolog of the murine Rosa26 locus, and the CCR5 chemokine receptor gene. Tools and techniques for the insertion of transgene (i.e., the exogenous DNA) into safe harbor locus are well known to one of skill in the art, see for example Papapetrou E P et al. Gene Insertion Into Genomic Safe Harbors for Human Gene Therapy. Mol Ther. (2016) 678-84.

Episomes are exogenous DNA that remains physically independent of the cell's endogenous chromosome or complement of chromosomes. Depending on its content and context, episomal DNA may replicate or synthesize messenger RNA (and, indirectly, protein), thus conferring on the cell novel biologic properties. Episomal concatemers derived from adeno-associated virus (AAV) vectors are thought not to replicate. Episomal iPSC Reprogramming Vectors or Enhanced Episomal Vectors (EEVs) can be employed for non-integrating, non-viral gene expression. Because they replicate in synchrony with the host cell, they are stably inherited and can be used for long-lasting expression-up to several months-without modifying the host genome.

Alternatively, gene delivery systems may use non-viral delivery using physical (carrier-free gene delivery) and chemical approaches (synthetic vector-based gene delivery). Such delivery systems are well known to one of skill in the art, see, for example, Zu, H., et al., Non-viral Vectors in Gene Therapy: Recent Development, Challenges, and Prospects. AAPSJ 23, 78 (2021), incorporated herein in its entirety by reference.

In various embodiments, iPSCs are prepared, and expanded using a culture system. Expanded iPSCs can be recovered from the culture for differentiating to embryoid bodies (EBs). EBs, created by differentiation of iPSCs, are three-dimensional aggregates of iPSCs and comprise the three (or alternatively two or one) embryonic germ layer(s) based on the differentiation method(s). Preparation of EBs is described, for example, in US 2019/0177695, which is hereby incorporated by reference in its entirety. In some embodiments, EBs prepared by differentiation of the iPSCs, are expanded in a bioreactor as described, for example, in Abecasis B. et al., Expansion of 3D human induced pluripotent stem cell aggregates in bioreactors: Bioprocess intensification and scaling-up approaches. J. of Biotechnol. 246 (2017) 81-93. EBs can be used to generate any desired cell type. Other methods, including a 3D suspension culture, for expansion or differentiation of EBs is described in WO 2020/086889, which is hereby incorporated by reference in its entirety.

In some embodiments, the process of producing a population comprising HSCs and/or HSPCs can comprise generating CD34+-enriched cells from the differentiated pluripotent stem cells (e.g., EBs) and inducing endothelial-to-hematopoietic differentiation. HSCs comprising relatively high frequency of LT-HSCs can be generated from the cell populations using various stimuli or factors, including mechanical, biochemical, metabolic, and/or topographical stimuli, as well as factors such as extracellular matrix, niche factors, cell-extrinsic factors, induction of cell-intrinsic properties; and including pharmacological and/or genetic means.

In some embodiments, the method comprises preparing endothelial cells with hemogenic potential from iPSCs, prior to induction of EHT. In some embodiments, the combined over-expression of GATA2/ETV2, GATA2/TAL1, or ER71/GATA2/SCL can lead to the formation of endothelial cells with hemogenic potential from PSC sources. In some embodiments, the method comprises overexpression of E26 transformation-specific variant 2 (ETV2) transcription factor in the iPSCs. Following CD34+ enrichment, HSCs are then generated from the endothelial cells using mechanical, biochemical, pharmacological and/or genetic stimulation or modification. ETV2 can be expressed by introduction of an encoding non-integrating episomal plasmid, for constitutive or inducible expression of ETV2, and for production of transgene-free hemogenic ECs. In some embodiments, ETV2 is expressed from an mRNA introduced into the iPSCs. mRNA can be introduced using any available method, including electroporation or lipofection. Differentiation of cells expressing ETV2 can comprise addition of VEGF-A. See, Wang K, et al., Robust differentiation of human pluripotent stem cells into endothelial cells via temporal modulation of ETV2 with mRNA. Sci. Adv. Vol. 6 (2020). Cells generated in this manner may be used for producing CD34+ cells and inducing EHT according to embodiments of this disclosure.

In embodiments, the endothelial-to-hematopoietic transition (EHT) is induced at Day 7 to Day 15 of iPSC differentiation. In various embodiments, the CD34+ population that are undergoing EHT can be harvested, that is, separated from other cells. In some embodiments, the endothelial-to-hematopoietic transition (EHT) generates an HSC population comprising one or more of long-term hematopoietic stem cells (LT-HSCs), short-term hematopoietic stem cells, and hematopoietic stem progenitor cells.

Conventionally, hematopoietic stem cells or hematopoietic lineages are prepared by differentiation of iPSCs to embryoid bodies up to day 8 to harvest CD34+ cells. CD34 is commonly used as a marker of hemogenic endothelial cells, hematopoietic stem cells, and hematopoietic progenitor cells. In accordance with aspects and embodiments of this disclosure, it is discovered that inducing endothelial-to-hematopoietic transition (EHT) of a CD34+ cell population (e.g., for at least two days, but not more than 12 days), and which can be derived from iPSCs-embryoid bodies, can be used for the ex vivo generation of superior HSCs and hematopoietic lineages.

In some embodiments, iPSC differentiation proceeds until cells are at least about 10% CD34+, or at least about 20% CD34+, or at least about 25% CD34+, or at least about 30% CD34+. In some embodiments, CD34+ enrichment and EHT may be induced at Day 8 to Day 14 of iPSC differentiation, such as for example, Day 8, Day 9, Day 10, Day 11, Day 12, Day 13, or Day 14. Differentiation of iPSCs can be according to known techniques. In some embodiments, iPSC differentiation involves factors such as, but not limited to, combinations of bFGF, Y27632, BMP4, VEGF, SCF, EPO, TPO, IL-6, IL-11, and/or IGF-1. In some embodiments, hPSCs are differentiated using feeder-free, serum-free, and/or GMP-compatible materials. In some embodiments, hPSCs are co-cultured with murine bone marrow-derived feeder cells such as OP9 or MS5 cell line in serum-containing medium. The culture can contain growth factors and cytokines to support differentiation of embryoid bodies or monolayer system. The OP9 co-culture system can be used to generate multipotent HSPCs, which can be optionally differentiated further to several hematopoietic lineages including T lymphocytes, B lymphocytes, megakaryocytes, monocytes or macrophages, and erythrocytes. See Netsrithong R. et al., Multilineage differentiation potential of hematoendothelial progenitors derived from human induced pluripotent stem cells, Stem Cell Research & Therapy Vol. 11 Art. 481 (2020). Alternatively, a step-wise process using defined conditions with specific signals can be used. For example, the expression of HOXA9, ERG, RORA, SOX4, and MYB in human PSCs favors the direct differentiation into CD34+/CD45+ progenitors with multilineage potential. Further, expression of factors such as HOXB4, CDX4, SCL/TAL1, or RUNX1a support the hematopoietic program in human PSCs. See Doulatov S. et al., Induction of multipotential hematopoietic progenitors from human pluripotent stem cells via re-specification of lineage-restricted precursors, Cell Stem Cell. 2013 Oct. 3; 13(4).

Where cell populations are described herein as having a certain phenotype it is understood that the phenotype represents a significant portion of the cell population, such as at least 25%, at least 40%, or at least about 50%, or at least about 60%, or at least about 75%, or at least about 80%, or at least about 90% of the cell population. Further, at various steps, cell populations can be enriched for cells of a desired phenotype, and/or depleted of cells of an undesired phenotype, such that cell population comprise at least about 75%, or at least about 80%, or at least about 90% of the desired phenotype. Such positive and negative selection methods are known in the art. For example, cells can be sorted based on cell surface antigens (including those described herein) using a fluorescence activated cell sorter, or magnetic beads which bind cells with certain cell surface antigens. Negative selection columns can be used to remove cells expressing undesired cell-surface markers. In some embodiments, cells are enriched for CD34+ cells (prior to and/or after undergoing EHT). In some embodiments, the cell population is cultured under conditions that promote expansion of CD34+ cells to thereby produce an expanded population of stem cells.

In various embodiments, the induction of endothelial-to-hematopoietic transition (EHT) comprises increasing the expression or activity of dnmt3b. For example, the induction of endothelial-to-hematopoietic transition (EHT) can comprise applying cyclic stretch to the iPSCs or cells derived from the iPSCs, such as CD34+ cells, endothelial cells (ECs), and hemogenic endothelial cells (HECs). In embodiments, the cyclic stretch is 2D, 3D, or 4D cyclic stretch.

For example, a cell population is introduced to a bioreactor that provides a cyclic-strain biomechanical stretching, as described in U.S. Pat. No. 11,162,073, which is hereby incorporated by reference in its entirety. The cyclic-strain biomechanical stretching can increase the activity or expression of Dnmt3b and/or Gimap6. In these embodiments, mechanical stretching means apply stretching forces to the cells, or to a cell culture surface having the cells (e.g., ECs or HECs) cultured thereon. For example, a computer controlled vacuum pump system or other means for providing a stretching force (e.g., the FlexCell™ Tension System, the Cytostretcher System) attached to flexible biocompatible and/or biomimetic surface can be used to apply cyclic 2D, 3D, or 4D stretch ex vivo to cells under defined and controlled cyclic strain conditions. For example, the applied cyclic stretch can be from about 1% to about 20% cyclic strain (e.g., about 6% cyclic strain) for several hours or days (e.g., about 7 days). In various embodiments, cyclic strain is applied for at least about one hour, at least about two hours, at least about six hours, at least about eight hours, at least about 12 hours, at least about 24 hours, at least about 48 hrs, at least about 72 hrs, at least about 96 hrs, at least about 120 hrs, at least about 144 hrs, or at least about 168 hrs.

Induction of EHT can be with any known process. In some embodiments, induction of EHT generates an HSC population comprising LT-HSCs. In some embodiments, EHT generates a cell population comprising HSPCs. In some embodiments, EHT generates HSCs and/or HSPCs through endothelial or hemogenic endothelial cell (HEC) precursors using mechanical, biochemical, pharmacological and/or genetic means (e.g., via stimulation, inhibition, and/or genetic modifications). In some embodiments, the EHT generates a stem cell population comprising one or more of long-term hematopoietic stem cells (LT-HSCs), short-term hematopoietic stem cells (ST-HSCs), and HSPCs. In various embodiments, EHT can be induced in the culture for from 2 days to 12 days, such as about 4 days to about 8 days (e.g., about 4 days, about 5 days, about 6 days, about 7 days, or about 8 days). In some embodiments, EHT is induced in the culture from about 5 days to about 7 days.

In various embodiments, CD34+ cells (e.g., the floater and/or adherent cells) are harvested from the culture undergoing endothelial-to-hematopoietic transition between Day 10 to Day 20 of iPSC differentiation, such as from Day 10 to Day 17, or from Day 12 to Day 15 of iPSC differentiation. In some embodiments, non-adherent cells are collected.

In some embodiments, the HSC and/or HSPC population or fraction thereof is differentiated to T cells or progenitors or derivatives thereof independent of the use of an agonist of a mechanosensitive receptor or a mechanosensitive channel, such as Yodal. In some embodiments, the use of an agonist of a mechanosensitive receptor or a mechanosensitive channel (e.g., Yodal) is optional. Thus, in some embodiments, CD34+ cells are enriched from a differentiated pluripotent stem cell population to prepare a CD34+-enriched population. Endothelial-to-hematopoietic transition of the CD34+-enriched cell population is induced for at least two days, but no more than 12 days in which the use of an agonist of a mechanosensitive receptor or a mechanosensitive channel such as Yodal, jedil, jedi2, or ssRNA40 is optional. The HSCs and/or HSPCs are differentiated to a progenitor T cell population or a T cell population (e.g., as described herein). In some embodiments, the endothelial-to-hematopoietic transition of the CD34+-enriched cell population is induced for at least for two days, and optionally further for about 4 hours, or about 8 hours, or about 12 hours, or about 16 hours, or about 20 hours, or about 24 hours, or about 2 days, or about 3 days, or about 4 days, or about 5 days, or about 6 days, or about 7 days, or about 8 days, or about 9 days, or about 10 days. The total EHT differentiation proceeds for no more than 12 days.

In embodiments, the induction of endothelial-to-hematopoietic transition (EHT) comprises Piezol activation. In embodiments, the Piezol activation is by contacting the iPSCs or cells derived from the iPSCs, with one or more Piezol agonists, which are optionally selected from Yodal, Jedi1, Jedi2, or analogues or derivatives thereof. For example, cells are contacted with an effective amount of an agonist of a mechanosensitive receptor or a mechanosensitive channel (e.g., Piezol agonist) that increases the activity or expression of Dnmt3b.

Yodal (2-[5-[[(2,6-Dichlorophenyl)methyl]thio]-1,3,4-thiadiazol-2-yl]-pyrazine) is a small molecule agonist developed for the mechanosensitive ion channel Piezol. Syeda R, Chemical activation of the mechanotransduction channel Piezol. eLife (2015). Yoda 1 has the following structure:

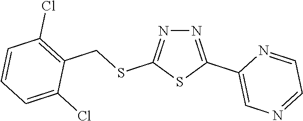 [SMILES: Clc1cccc(Cl)c1CSc1nnc(-c2cnccn2)s1]

Derivatives of Yodal can be employed in various embodiments. For example, derivatives comprising a 2,6-dichlorophenyl core are employed in some embodiments. Exemplary agonists are disclosed in Evans E L, et al., Yodal analogue (Dookul) which antagonizes Yodal-evoked activation of Piezol and aortic relaxation, British J. of Pharmacology 175(1744-1759): 2018. Still otherPiezol agonist include Jedi1, Jedi2, single-stranded (ss) RNA (e.g., ssRNA40) and derivatives and analogues thereof. See Wang Y., et al., A lever-like transduction pathway for long-distance chemical-and mechano-gating of the mechanosensitive Piezol channel. Nature Communications (2018) 9:1300; Sugisawa, et al., RNA Sensing by Gut Piezol Is Essential for Systemic Serotonin Synthesis, Cell, Volume 182, Issue 3, 2020, Pages 609-624, which are hereby incorporated by reference in their entireties. These Piezol agonists are commercially available. In various embodiments, the effective amount of the Piezol agonist or derivative is in the range of about 1 μM to about 500 M, or about 5 M to about 200 VM, or about 5 μM to about 100 μM, or in some embodiments, in the range of about 25 μM to about 150 μM, or about 25 μM to about 100 μM, or about 25 M to about 50 VM. Alternatively, single-stranded (ss) RNA (e.g., ssRNA40), and derivatives and analogues thereof, can be used for Piezol activation.

Alternatively or in addition, EHT is stimulated by Trpv4 activation. The Trpv4 activation can be by contacting cells (e.g., CD34-enriched cells, ECs, or HECs) with one or more Trpv4 agonists, which are optionally selected from GSK1016790A, 4alpha-PDD, or analogues and/or derivatives thereof. In embodiments, the Trpv4 activation is by contacting the iPSCs or cells derived from iPSCs thereof with one or more Trpv4 agonists, which are optionally selected from GSK1016790A, 4alpha-PDD, or analogues or derivatives thereof.

In various embodiments, pharmacological Piezol activation is applied to CD34+ cells (i.e., CD34+-enriched cells). In certain embodiments, pharmacological Piezol activation may further be applied to iPSCs, embryoid bodies (EBs), endothelia cells (ECs), hemogenic endothelial cells (HECs), HSCs, hematopoietic progenitors, as well as hematopoietic lineage(s). In certain embodiments, Piezol activation is applied at least to EBs generated from iPSCs, CD34+ cells isolated from EBs, and/or combinations thereof, which in accordance with various embodiments, allows for superior generation of HSCs as compared to other methods for inducing EHT.

Alternatively or in addition, the activity or expression of Dnmt3b can be increased directly in the cells, e.g., in CD34+-enriched cells. For example, mRNA expression of Dnmt3b can be increased by delivering Dnmt3b-encoding transcripts to the cells, or by introducing a Dnmt3b-encoding transgene, or a transgene-free method, not limited to introducing a non-integrating episome to the cells. In some embodiments, gene editing is employed to introduce a genetic modification to Dnmt3b expression elements in the cells, such as, but not limited to, to increase promoter strength, ribosome binding, RNA stability, and/or impact RNA splicing.

In some embodiments, the method comprises increasing the activity or expression of Gimap6 in the cells, alone or in combination with Dnmt3b and/or other genes that are up- or down regulated upon cyclic strain or Piezol activation. To increase activity or expression of Gimap6, Gimap6-encoding mRNA transcripts can be introduced to the cells, transgene-free approaches can also be employed, including but not limited, to introducing an episome to the cells; or alternatively a Gimap6-encoding transgene. In some embodiments, gene editing is employed to introduce a genetic modification to Gimap6 expression elements in the cells (such as one or more modifications to increase promoter strength, ribosome binding, RNA stability, or to impact RNA splicing).

In embodiments of this disclosure employing mRNA delivery to cells, known chemical modifications can be used to avoid the innate-immune response in the cells. For example, synthetic RNA comprising only canonical nucleotides can bind to pattern recognition receptors and can trigger a potent immune response in cells. This response can result in translation block, the secretion of inflammatory cytokines, and cell death. RNA comprising certain non-canonical nucleotides can evade detection by the innate immune system, and can be translated at high efficiency into protein. See U.S. Pat. No. 9,181,319, which is hereby incorporated by reference, particularly with regard to nucleotide modification to avoid an innate immune response.

In some embodiments, expression of Dnmt3b and/or Gimap6 is increased by introducing a transgene into the cells, which can direct a desired level of overexpression (with various promoter strengths or other selection of expression control elements). Transgenes can be introduced using various viral vectors or transfection reagents (including Lipid Nanoparticles) as are known in the art. In some embodiments, expression of Dnmt3b and/or Gimap6 is increased by a transgene-free method (e.g., episome delivery). In some embodiments, expression or activity of Dnmt3b and/or Gimap6 or other genes disclosed herein are increased using a gene editing technology, for example, to introduce one or more modifications to increase promoter strength, ribosome binding, or RNA stability.

In still other embodiments, the CD34+-enriched cells are cultured with an inhibitor of histone methyltransferase EZH1 during EHT. Alternatively, EZH1 is partially or completely deleted or inactivated or is transiently silenced in the stem cell population. See WO 2018/048828, which is hereby incorporated by reference in its entirety.

In various embodiments, CD34+ cells (e.g., the floater and/or adherent cells) are harvested from the culture undergoing endothelial-to-hematopoietic transition between Day 8 to Day 15 of iPSC differentiation. In some embodiments, CD34+ floater cells are harvested during days 8 to 15 of iPSC differentiation.

In various embodiments, the HSCs or CD34+-enriched cells are further expanded. For example, the HSCs or CD34+-enriched cells can be expanded according to methods disclosed in U.S. Pat. Nos. 8,168,428; 9,028,811; 10,272,110; and 10,278,990, which are hereby incorporated by reference in their entireties. In some embodiments, ex vivo expansion of HSCs or CD34+-enriched cells employs prostaglandin E₂ (PGE2) or a PGE₂ derivative. In some embodiments of this disclosure, the HSCs comprise at least about 0.01% LT-HSCs, or at least about 0.05% LT-HSCs, or at least about 0.1% LT-HSCs, or at least about 0.5% LT-HSCs, or at least about 1% LT-HSCs.

Hematopoietic stem cells (HSCs) which give rise to erythroid, myeloid, and lymphoid lineages, can be identified based on the expression of CD34 and the absence of lineage specific markers (termed Lin−). In some embodiments, a population of stem cells comprising HSCs are enriched, for example, as described in U.S. Pat. No. 9,834,754, which is hereby incorporated by reference in its entirety. For example, this process can comprise sorting a cell population based on expression of one or more of CD34, CD90, CD38, and CD43. A fraction can be selected for further differentiation that is one or more of CD34⁺, CD90⁻, CD38⁻, and CD43⁻. In some embodiments, the stem cell population for differentiation to a hematopoietic lineage is at least about 80% CD34⁺, or at least about 90% CD34⁺, or at least about 95% CD34⁺.

In some embodiments, the stem cell population, or CD34+-enriched cells or fraction thereof, or derivative population are expanded as described in US 2020/0308540, which is hereby incorporated by reference in its entirety. For example, the cells are expanded by exposing the cells to an aryl hydrocarbon receptor antagonist including, for example, SR1 or an SR1-derivative. See also, Wagner et al., Cell Stem Cell 2016; 18(1):144-55 and Boitano A., et al., Aryl Hydrocarbon Receptor Antagonists Promote the Expansion of Human Hematopoietic Stem Cells. Science 2010 Sep. 10; 329(5997): 1345-1348.

In some embodiments, the compound that promotes expansion of CD34⁺ cells includes a pyrimidoindole derivative including, for example, UM171 or UM729 (see US 2020/0308540, which is hereby incorporated by reference).

In some embodiments, the stem cell population or CD34+-enriched cells are further enriched for cells that express Periostin and/or Platelet Derived Growth Factor Receptor Alpha (pdgfra) or are modified to express Periostin and/or pdgfra, as described in WO 2020/205969 (which is hereby incorporated by reference in its entirety). Such expression can be by delivering encoding transcripts to the cells, or by introducing an encoding transgene, or a transgene-free method, not limited to introducing a non-integrating episome to the cells. In some embodiments, gene editing is employed to introduce a genetic modification to expression elements in the cells, such as to modify promoter activity or strength, ribosome binding, RNA stability, or impact RNA splicing.

In embodiments, the HSCs are further differentiated to a hematopoietic lineage for therapy. In embodiments, the hematopoietic lineage is selected from progenitor-T cells, T lymphocytes, and Natural Killer cells. Other lineages that can be produced and used for therapy include common myeloid progenitor (CMP) or common lymphoid progenitor (CLP) cells. CMPs give rise to progenies, such as, red blood cells/erythrocytes, platelets, mast cells, osteoclasts, granulocytes, monocyte-macrophages, and dendritic cells. CLPs give rise to progenies such as T-cells/T-lymphocytes, B-cells/B-lymphocytes, NK-cells/natural killer cells, and dendritic cells.

For example, an HSC cell population or cells harvested therefrom can be cultured with a Notch ligand (partial or full), SHH, extracellular matrix component(s), and/or combinations thereof, ex vivo, to differentiate HSCs to CD7+ progenitor T cells, and optionally to a T cell lineage or other lineage (e.g., NK cell).

The term “Notch ligand” as used herein refers to a ligand capable of binding to a Notch receptor polypeptide present in the membrane of a hematopoietic stem cell or progenitor T cell. The Notch receptors include Notch-1, Notch-2, Notch-3, and Notch-4. Notch ligands typically have a DSL domain (D-Delta, S-Serrate, and L-Lag2) comprising 20-22 amino acids at the amino terminus, and from 3 to 8 EGF repeats on the extracellular surface. In various embodiments, the Notch ligand comprises at least one of Delta-Like-1 (DLL1), Delta-Like-4 (DLL4), SFIP3, or a functional portion thereof. A key signal that is delivered to incoming lymphocyte progenitors by the thymus stromal cells in vivo is mediated by DL4, which is expressed by cortical thymic epithelial cells.

In some embodiments, the Notch ligand is a DLL4 having one or more affinity enhancing mutations, such as one or more (or all) of: G28S, F107L, I143F, H194Y, L206P, N257P, T271L, F280Y, S301R and Q305P, with respect to hDLL4. See Gonzalez-Perez, et al., Affinity-matured DLL4 ligands as broad-spectrum modulators of Notch signaling, Nature Chemical Biology (2022).

The earliest intrathymic progenitors express high levels of CD34 and CD7, do not express CD1a, and are triple-negative (TN) for mature T cell markers: CD4, CD8, and CD3. Commitment to the T cell lineage is associated with the expression of CD1a by CD7-expressing pro-thymocytes. Thus, immature stages of T-cell development are typically delineated as CD34⁺CD1a⁻ (most immature) and CD34⁺CD1a⁺ cells. The transition from CD34⁺CD7⁺CD1a⁻ to CD34⁺CD7⁺CD1a⁺ by early thymocytes is associated with T-cell commitment. CD34⁺CD7⁺CD1a⁺ cells are likely T-lineage restricted. Following this stage, thymocytes progress to a CD4 immature single positive stage, at which point CD4 is expressed in the absence of CD8. Thereafter, a subset of the cells differentiates to the CD4⁺CD8⁺ double positive (DP) stage. Finally, following TCRα rearrangement, TCRαβ-expressing DP thymocytes undergo positive and negative selection, and yield CD4⁺CD8⁻ and CD4⁻CD8⁺ single positive (SP) T-cells.

In some embodiments, progenitor T cells are isolated by enrichment for CD7 expression. In some embodiments, progenitor T cells are expanded as described in US 2020/0308540, which is hereby incorporated by reference in its entirety. For example, the cells may be expanded by exposing the cells to an aryl hydrocarbon receptor antagonist including, for example, SR1 or an SR1-derivative. See also, Wagner et al., Cell Stem Cell 2016; 18(1):144-55. In some embodiments, the compound that promotes expansion includes a pyrimidoindole derivative including, for example, UM171 or UM729 (see US 2020/0308540, which is hereby incorporated by reference).

Differentiation to progenitor T cells can further include in some embodiments the presence of stem cell factor (SCF), Flt3L and interleukin (IL)-7. In various embodiments, CD7+ progenitor T cells created express CD1a. The CD7+ progenitor T cells do not express CD34 or express a diminished level of CD34 compared to the HSC population. In some embodiments, the CD7+ progenitor T cells (or a portion thereof) further express CD5. Accordingly, the phenotype of the progenitor T cells may be CD7⁺CD1a⁺. In some embodiments, the phenotype of the progenitor T cells is CD7⁺CD5⁺. In some embodiments, the progenitor T cells are CD7⁺CD1a⁺CD5⁺, and optionally CD34⁺.

In some embodiments, the Notch ligand is an anti-Notch (agonistic) antibody that can bind and engage Notch signaling. In some embodiments, the antibody is a monoclonal antibody (including a human or humanized antibody), a single chain antibody (scFv), a nanobody, or other antibody fragment or antigen-binding molecule capable of activating the Notch signaling pathway.

In some embodiments, the Notch ligand is a Delta family Notch ligand. The Delta family ligand in some embodiments is Delta-1 (Genbank Accession No. AF003522, Homo sapiens), Delta-like 1 (DLL1, Genbank Accession No. NM_005618 and NP_005609, Homo sapiens; Genbank Accession No. X80903, 148324, M. musculus), Delta-4 (Genbank Accession No. AF273454, BAB18580, Mus musculus; Genbank Accession No. AF279305, AAF81912, Homo sapiens), and/or Delta-like 4 (DLL4; Genbank Accession. No. Q9NR61, AAF76427, AF253468, NM_019074, Homo sapiens; Genbank Accession No. NM 019454, Mus musculus). Notch ligands are commercially available or can be produced, for example, by recombinant DNA techniques.

In various embodiments, the Notch ligands are soluble, and are optionally immobilized on microparticles or nanoparticles, which are optionally paramagnetic to allow for magnetic enrichment or concentration processes. In still other embodiments, the Notch ligands are immobilized on a 2D or 3D culture surface, optionally with other adhesion molecules such as VCAM-1. See US 2020/0399599, which is hereby incorporated by reference in its entirety. In other embodiments, the beads or particles are polymeric (e.g., polystyrene or PLGA), gold, iron dextran, or constructed of biological materials, such as particles formed from lipids and/or proteins. In various embodiments, the particle has a diameter or largest dimension of from about 0.01 μm (10 nm) to about 500 μm (e.g., from about 1 μm to about 7 μm). In still other embodiments, polymeric scaffolds with conjugated ligands can be employed, as described in WO 2020/131582, which is hereby incorporated by reference in its entirety. For example, scaffold can be constructed of polylactic acid, polyglycolic acid, PLGA, alginate or an alginate derivative, gelatin, collagen, agarose, hyaluronic acid, poly(lysine), polyhydroxybutyrate, poly-epsilon-caprolactone, polyphosphazines, poly(vinyl alcohol), poly(alkylene oxide), poly(ethylene oxide), poly(allylamine), poly(acrylate), poly(4-aminomethylstyrene), pluronic polyol, polyoxamer, poly(uronic acid), poly(anhydride), poly(vinylpyrrolidone), and any combination thereof. In some embodiments, the scaffold comprises pores having a diameter between about 1 μm and 100 μm.

In some embodiments, the C-terminus of the Notch ligand is conjugated to the selected support. In some embodiments, this can include adding a sequence at the C-terminal end of the Notch ligand that can be enzymatically conjugated to the support, for example, through a biotin molecule. In another embodiment, a Notch ligand-Fc fusion is prepared, such that the Fc segment can be immobilized by binding to protein A or protein G that is conjugated to the support. Of course, any of the known protein conjugation methods can be employed.

Thus, in various embodiments, the Notch ligand is immobilized, functionalized, and/or embedded in 2D or 3D culture system. The Notch ligand may be incorporated along with a component of extracellular matrix, such as one or more selected from fibronectin, RetroNectin, and laminin. In some embodiments, the Notch ligand and/or component of extracellular matrix are embedded in inert materials providing 3D culture conditions. Exemplary materials include, but are not limited to, cellulose, alginate, and combinations thereof. In some embodiments, the Notch ligand, a component of extracellular matrix, or combinations thereof, are in contact with culture conditions providing topographical patterns and/or textures (e.g., roughness) to cells conducive to differentiation and/or expansion.

In some embodiments, the population comprising HSCs and/or HSPCs are differentiated to progenitor T cells by culture in medium comprising TNF-α and/or antagonist of aryl hydrocarbon/dioxin receptor (SR1), and in the presence of Notch ligand. See US 2020/0390817, US 2021/0169934, and US 2021/0169935, which are hereby incorporated by reference in its entirety. In some embodiments the HSCs are cultured in a medium comprising TNF-α, IL-7, thrombopoietin (TPO), Flt3L, and stem cell factor (SCF), and optionally SRI, in the presence of an immobilized Delta-Like-4 ligand and a fibronectin fragment. In some embodiments, the cells are cultured with RetroNectin, which is a recombinant human fibronectin containing three functional domains: the human fibronectin cell-binding domain (C-domain), heparin-binding domain (H-domain), and CS-1 sequence domain. In some embodiments, cells are cultured in the presence of an immobilized Delta-Like-4 ligand and a RetroNectin. In some embodiments, cells are cultured in the presence of an immobilized Delta-Like-4 ligand, TNF-alpha, and a RetroNectin. In some embodiments, cells are cultured in the presence of an immobilized Delta-Like-1 ligand and a RetroNectin. In some embodiments, cells are cultured in the presence of SFIP3 and RetroNectin. In some embodiments, cells are cultured in the presence of an immobilized Delta-Like-4 ligand and SHH molecules and/or functional derivatives thereof. Exemplary fibronectin fragments include one or more RGDS, CS-1, and heparin-binding motifs. Fibronectin fragments can be free in solution or immobilized to the culture surface or on particles. In some embodiments, cells are cultured for 5 to 7 days to prepare CD7+ progenitor T cells.

In various embodiments, the method produces progenitor T cells, or a T cell lineage, by culturing the HSC population with the Notch ligand (including any of the embodiments described above) with or without component(s) extracellular matrix, and optionally adding TNF-alpha to the culture at certain stages of differentiation. Thus, cells created in some embodiments are progenitor or precursor cells committed to the T cell lineage (“progenitor T cells”). In some embodiments, the cells are CD7⁺ progenitor T cells. In some embodiments, the cells are CD25⁺ immature T cells, or cells that have undergone CD4 or CD8 lineage commitment. In some embodiments, the cells are CD4⁺CD8⁺ double positive (DP), CD4⁻CD8⁺, or CD4⁺CD8⁻. In some embodiments, the cells are single positive (SP) cells that are CD4⁻CD8⁺ or CD4⁺CD8⁻ and TCR^{hi}. In some embodiments, the cells are TCRαβ⁺ and/or TCRγΔ⁺. In various embodiments, the cells are CD3⁺.

The adoptive transfer of progenitor T cells is a strategy for enhancing T cell reconstitution. Progenitor T cells are developmentally immature and undergo positive and negative selection in the host thymus. Thus, they become restricted to the recipient's major histocompatibility complex (MHC) yielding host tolerant T cells that can bypass the clinical challenges associated with graft-versus-host disease (GVHD). Importantly, engraftment with progenitor T cells restores the thymic architecture and improves subsequent thymic seeding by HSC-derived progenitors. In addition to its intrinsic regenerative medicine properties, progenitor T cells can also be engineered with T cell receptors (TCRs) and chimeric antigen receptors (CARs) (via either gene or mRNA delivery) to confer specificity to tumor-associated antigens.

In various embodiments, the progenitor T cells are further cultured under suitable conditions to generate cells of a desired T cell lineage, including with one or more Notch ligands. For example, the cells can be cultured in the presence of one or more Notch ligands as described for a sufficient time to form cells of the T cell lineage. In some embodiments, stem cells or progenitor T cells are cultured in suspension with soluble Notch ligand orNotch ligand conjugated to particles or other supports, or Notch ligand expressing cells. In some embodiments, the progenitor T cells or stem cells are cultured in suspension or in adherent format in a bioreactor, optionally a closed or a closed, automated bioreactor, with a soluble or conjugated Notch ligand in suspension. One or more cytokines, extracellular matrix component(s), and thymic niche factor(s) that promote commitment and differentiation to the desired T cell lineage may also be added to the culture or reactor. Such cytokines or factors are known in the art. In various embodiments, the population comprising HSCs and/or HSPCs is cultured with the Notch ligand for about 4 to about 21 days, or from about 6 to about 18 days, or from about 7 to about 14 days to generate progenitor T cells. In some embodiments, the stem cell population or derivative thereof is cultured for at least about 21 days or at least about 28 days to generate mature T cell lineages or NK cells.

In various embodiments, the HSC population is cultured in an artificial thymic organoid (ATO). See, Hagen, M. et al. (2019). The ATO will include culture of HSCs (or aggregates of HSCs) with a Notch ligand-expressing stromal cell line in serum-free conditions. The artificial thymic organoid is a 3D system, inducing differentiation of hematopoietic precursors to naive CD3⁺CD8⁺ and CD3⁺CD4⁺ T cells.

In various embodiments, the method comprises generating a derivative of the progenitor T cells or generating a T cell lineage from the progenitor T cells. In certain embodiments, the derivative of the progenitor T cell or T cell lineage expresses CD3 and a T cell receptor. In some embodiments, the T cell lineage is CD8⁺ and/or CD4⁺. For example, T cells lineages can include one or more of CD8⁺CD4⁻, CD8⁻CD4⁺, CD8⁺CD4⁺, and CD8⁻CD4⁻ cells. In some embodiments, the iPSCs, CD34+ cells, or derivatives thereof are modified to express a chimeric antigen receptor (CAR) at progenitor-T, T-cell, and/or NK cell level.

In some embodiments, the derivative of the progenitor T cell is a natural killer (NK) cell. In some embodiments, NK cells are generated from progenitor T cells as described in U.S. Pat. No. 10,266,805, which is hereby incorporated by reference in its entirety. For example, the progenitor T cells can give rise to NK cells when cultured with IL-15. In some embodiments, the NK cell expresses a CAR, based on gene editing of iPSC, embryonic bodies, hCD34+ cells, or NK cells, or via mRNA expression in NK cells.

In some aspects and embodiments, the modified HSCs replace normal cells that were destroyed by antibodies, T-cell therapy (e.g., CAR-T therapy), or NK cell therapy (e.g., CAR-NK). The HSCs and progenitors thereof help the bone marrow recover and make healthy cells, e.g., myeloid cells in AML treatment. HSC rescue allows more targeted therapy to be given to a patient so that more cancer cells are killed.

In various embodiments, the modified HSCs/HSPCs of the present disclosure may be used to treat or ameliorate a disease or a disorder, such as treating a hematopoietic malignancy. Non-limiting examples of hematopoietic malignancies include, cytogenetically normal acute myeloid leukemia (CN-AML), acute myeloid leukemia (AML), acute lymphoid leukemia, chronic myelogenous leukemia, acute lymphoblastic leukemia or chronic lymphoblastic leukemia, chronic lymphoid leukemia, Hodgkin's lymphoma, non-Hodgkin's lymphoma, multiple myeloma or refractory or relapsing versions thereof. In some embodiments, the subject has, is, or will undergo a targeted therapy, for example, a therapy that targets hematopoietic cells (or lineage thereof).

In embodiments, the administration of the HSCs and/or HSPCs is performed following myeloablative, non-myeloablative, or immunotoxin-based (e.g., anti-c-Kit, anti-CD45, etc.) conditioning regimes.

In various embodiments, the modified HSCs and/or HSPCs of the disclosure (or cell lineage therefrom) is administered in connection with therapeutic applications of CAR-T therapy. Generally, the CAR-T cells target a cancer antigen that has reduced or eliminated expression in the modified HSCs. Exemplary CAR-T cells include CD33-specific CAR-T cells, CD7-specific CAR-T cells, CD8-specific CAR-T cells, CD19-specific CAR-T cells, CD20-specific CAR-T cells, CD22-specific CAR-T cells, CD123-specific CAR-T cells, CD125-specific CAR-T cells, CD133-specific CAR-T cells, and CD371-specific CAR-T cells. In some embodiments, the HSCs of the disclosure are administered in connection with CAR-NK cell therapy.

In various embodiments, the HSCs and/or HSPCs or cell lineages derived therefrom (e.g., T cells or NK cells, including CAR-T cells or CAR-NK cells) are administered in connection with treatment of non-hematological malignancies, where the targeted antigen is expressed (even if at low levels) in normal hematopoietic cells or lineages. Such cancer-associated antigens include: Human epidermal growth factor receptor 2 (HER2) (e.g., for ovarian cancer, breast cancer, glioblastoma, colon cancer, osteosarcoma, and medulloblastoma); Epidermal growth factor receptor (EGFR) (e.g., for non-small cell lung cancer, epithelial carcinoma, and glioma); Mesothelin (e.g., for mesothelioma, ovarian cancer, and pancreatic adenocarcinoma); Prostate-specific membrane antigen (PSMA) (e.g., for prostate cancer); Carcinoembryonic antigen (CEA) (e.g., for pancreatic adenocarcinoma, breast cancer, and colorectal carcinoma); Glypican-3 (e.g., for hepatocellular carcinoma); Variant III of the epidermal growth factor receptor (EGFRvIII) (e.g., for glioblastoma); Disialoganglioside 2(GD2) (e.g., for neuroblastoma and melanoma); Carbonic anhydrase IX(CAIX) (e.g., for renal cell carcinoma); Interleukin-13Ra2 (e.g., for glioma); Fibroblast activation protein (FAP) (e.g., for malignant pleural mesothelioma); L1 cell adhesion molecule (L1-CAM) (e.g., for neuroblastoma, melanoma, and ovarian); Cancer antigen 125 (CA 125) (e.g., for epithelial ovarian cancer); Cluster of differentiation 133 (CD 133) (e.g., for glioblastoma and cholangiocarcinoma, adenocarcinoma); Cancer/testis antigen 1B (CTAG1B) (e.g., for melanoma and ovarian cancer); Mucin 1 (e.g., for seminal vesicle cancer); Folate receptor-a (FR-a) (e.g., for ovarian cancer); and Growth factor receptor selected from one or more of ErbB1, ErbB2, ErbB3, or ErbB4, IGF1R, IGF2R, TPR I-II, VEGFR1, VEGFR2, VEGFR3, PDGFR (α/β), and FGFR1 through 4.

The modified HSCs according to this disclosure can be used (e.g., in methods of treatment) in connection with FDA approved CAR-T therapy, such as, Tisagenlecleucel, also known as tisa-cel (KYMRIAH), Axicabtagene ciloleucel, also known as axi-cel (YESCARTA), Brexucabtagene autoleucel, also known as brexu-cel (TECARTUS), Lisocabtagene maraleucel, also known as liso-cel (BREYANZI), Idecabtagene vicleucel, also known as ide-cel (ABECMA), Ciltacabtegene autoleucel, also known as cilta-cel (CARVYKTI) or any other CAR-T therapy which damage the normal cells (e.g., normal hematopoietic cells) during their therapeutical applications.

In various embodiments, the modified HSC of the invention is administered to mitigate the killing of normal cells or adverse effects caused by therapeutic applications of antibody therapy which target cancer associated antigens selected from one or more of growth factor receptors, ErbB1, ErbB2, ErbB3, or ErbB4, IGF1R, IGF2R, TPR I-II, VEGFR1, VEGFR2, VEGFR3, PDGFR (α/β) or FGFR1, FGFR2, FGFR3, or FGFR4 or antibodies that target checkpoint proteins. Antibodies targeting such antigens include, but are not limited to alefacept (AMEVIVE), alemtuzumab (CAMPATH), belimumab (BENLYSTA), cetuximab (ERBITUX), daclizumab (ZENAPAX, ZINBRYTA), denosumab (PROLIA, XGEVA), efalizumab (RAPTIVA), ipilimumab (YERVOY), natalizumab (TYSABRI), nivolumab (OPDIVO), olaratumab (LARTRUVO), panitumumab (VECTMIX), pembrolizumab (KEYTRUDA), rituximab (RITUXAN), trastuzumab (HERCEPTIN), bevacizumab, or a combination thereof. Furthermore, the antibodies may be selected from anti-estrogen receptor antibody, anti-progesterone receptor antibody, anti-p53 antibody, anti-EGFR antibody, anti-cathepsin D antibody, anti-Bcl-2 antibody, anti-E-cadherin antibody, anti-CA125 antibody, anti-CA15-3 antibody, anti-CA19-9 antibody, anti-c-erbB-2 antibody, anti-P-glycoprotein antibody, anti-CEA antibody, anti-retinoblastoma protein antibody, anti-ras oncoprotein antibody, anti-Lewis X antibody, anti-Ki-67 antibody, anti-PCNA antibody, anti-CD3 antibody, anti-CD4 antibody, anti-CD5 antibody, anti-CD7 antibody, anti-CD8 antibody, anti-CD9/p24 antibody, anti-CD1-antibody, anti-CD11c antibody, anti-CD13 antibody, anti-CD14 antibody, anti-CD15 antibody, anti-CD19 antibody, anti-CD20 antibody, anti-CD22 antibody, anti-CD23 antibody, anti-CD30 antibody, anti-CD31 antibody, anti-CD33 antibody, anti-CD35 antibody, anti-CD38 antibody, anti-CD39 antibody, anti-CD41 antibody, anti-LCA/CD45 antibody, anti-CD45RO antibody, anti-CD45RA antibody, anti-CD71 antibody, anti-CD95/Fas antibody, anti-CD99 antibody, anti-CD100 antibody, anti-S-100 antibody, anti-CD106 antibody, anti-ubiquitin antibody, anti-c-myc antibody, anti-cytokeratin antibody, anti-lambda light chains antibody, anti-melanosomes antibody, anti-prostate specific antigen antibody, anti-tau antigen antibody, anti-fibrin antibody, anti-keratins antibody, and anti-Tn-antigen antibody or any other antibody based therapy which damage the normal cells during their therapeutical applications. In such embodiments, the antigen targeted is reduced in expression or deleted in HSCs or cell population derived therefrom.

The composition comprising the modified HSCs and/or HSPCs of this disclosure may further comprise a pharmaceutically acceptable excipient or a carrier. Such excipients or carrier solutions also can contain buffers, diluents, and other suitable additives. A buffer refers to a solution or liquid whose chemical makeup neutralizes acids or bases without a significant change in pH. Examples of buffers envisioned by the invention include, but are not limited to, normal/physiologic saline (0.9% NaCl), 5% dextrose in water (D5W), Dulbecco's phosphate buffered saline (PBS), Ringer's solution. The composition may comprise a vehicle suitable for intravenous infusion or other administration route and may include a suitable cryoprotectant. An exemplary carrier is DMSO (e.g., about 10% DMSO). Cell compositions may be provided in implantable devices (e.g., scaffolds) or in bags or in vials, tubes or a container in an appropriate volume and stored frozen until use.

In other aspects, the present disclosure provides a cell population, or pharmaceutically acceptable composition thereof, produced by the method described herein. In various embodiments and as described herein, the HSC and/or HSPC population: is derived from iPSCs; has a genetically-disrupted expression of one or more endogenous genes that are cancer-associated antigens; proliferates in vivo but does not exhibit uncontrolled growth or tumor formation in vivo; and differentiates in vivo to reconstitute hematopoietic lineages.

For example, in an aspect, the disclosure provides an HSC population that has a genetically-disrupted expression of one or more endogenous genes that are tumor-associated antigens; and wherein the iPSCs are: HLA-A^{neg}, homozygous for both HLA-B and HLA-C, HLA-DPB1^{neg}, HLA-DQB1^{neg} and homozygous for HLA-DRB1. As described elsewhere herein, the tumor-associated antigen is expressed or overexpressed in one or more of cytogenetically normal acute myeloid leukemia (CN-AML), acute myeloid leukemia (AML), acute lymphoid leukemia, chronic myelogenous leukemia, acute lymphoblastic leukemia, chronic lymphoblastic leukemia, chronic lymphoid leukemia, Hodgkin's lymphoma, non-Hodgkin's lymphoma, and multiple myeloma. In various embodiments, the endogenous gene is expressed on immune cells, and may be expressed on myeloid cells, thymocytes (including T cells or B cells). In various embodiments, at least one of the endogenous genes is selected from CD33, CD19, CD7, CD123, and CD371.

In various embodiments, the composition for cell therapy (e.g., comprising an HSC population described herein) comprises the desired cell population and a pharmaceutically acceptable vehicle. The pharmaceutical composition may comprise at least about 10² cells, or at least about 10³, or at least about 10⁴, or at least about 10⁵, or at least about 10⁶, or at least about 10⁷, or at least about 10⁸ cells, or at least about 10⁹ cells, or at least about 10¹⁰ cells, or at least about 10¹¹ cells, or at least about 10¹² cells, or at least about 10¹³ cells, or at least about 10¹⁴ cells. For example, in some embodiments, the pharmaceutical composition is administered, comprising T progenitors of from about 100,000 to about 400,000 cells per kilogram (e.g., about 200,000 cells/kg). In other embodiments, cells are administered at from about 10⁵ to about 5×10⁵ cells per kilogram (e.g., about 2.5×10⁵ cells/kg), or from about 10⁶ to about 5×10⁶ cells per kilogram (e.g., about 2.5×10⁶ cells/kg), or from about 5×10⁶ to about 10⁷ cells per kilogram (e.g., about 5×10⁶ cells/kg) or from about 10⁷ to about 10⁸ cells per kilogram (e.g., about 5×10⁷ cells/kg) or from about 10⁸ to about 10⁹ cells per kilogram (e.g., about 5×10⁸ cells/kg) or from about 10⁹ to about 10¹⁰ cells per kilogram or from about 10¹⁰ to about 10¹¹ cells or from about 10¹¹ to about 10¹² cells per kilogram or from about 10¹² to about 10¹³ cells per kilogram or from about 10¹³ to about 10¹⁴ cells per kilogram of a recipient's body weight.

Routes of administration of the cells (e.g., modified HSCs and/or HSPCs or progenies thereof) could be by any suitable means, including but not limited to, parenteral routes. Parenteral infusions include intravenous and intraarterial administration. In addition, the cells (e.g., modified HSCs and/or HSPCs or progenies thereof) may suitably be administered by pulse infusion, e.g., with declining doses of the cells (e.g., modified HSCs and/or HSPCs or progenies thereof). In some embodiments the dosing is given by injections, for example via intravenous injections.

As used herein, the term “about” means 10% of the associated numerical value.

Certain aspects and embodiments of this disclosure are further described with reference to the following examples.

### EXAMPLES

### Example 1—ETV2 Over-Expression Increases the Yield of Hemogenic Endothelial Cells and Enhances the CD34+ Cell Formulation During iPSC Differentiation but does not Affect Pluripotency

### Methods

iPSCs were developed from hCD34+ cells by episomal reprogramming as known in the art and essentially as described in Yu, et al. Induced pluripotent stem cell lines derived from human somatic cells, Science 318, 1917-1920, (2007); and J. Yu, et al. Human induced pluripotent stem cells free of vector and transgene sequences. Science 324, 797-801, (2009). Embryoid Bodies and hemogenic endothelium differentiation was performed essentially as described in: R. Sugimura, et al., Haematopoietic stem and progenitor cells from human pluripotent stem cells. Nature 545, 432-438, (2017); C. M. Sturgeon, et al, Wnt signaling controls the specification of definitive and primitive hematopoiesis from human pluripotent stem cells. Nat Biotechnol 32, 554-561, (2014); J. Yu, et al. Induced pluripotent stem cell lines derived from human somatic cells. Science 318, 1917-1920, (2007); and J. Yu, et al. Human induced pluripotent stem cells free of vector and transgene sequences. Science 324, 797-801, (2009).

Briefly, hiPSC were dissociated and resuspended in media supplemented with L-glutamine, penicillin/streptomycin, ascorbic acid, human holo-Transferrin, monothioglycerol, BMP4, and Y-27632. Next, cells were seeded in 10 cm dishes (EZSPHERE or low attachment plate) for the EB formation. On Day 1, bFGF and BMP4 were added to the medium. On Day 2, the media was replaced with a media containing SB431542, CHIR99021, bFGF, and BMP4. On Day 4, the cell media was replaced with a media supplemented with VEGF and bFGF. On day 6, the cell media was replaced with a media supplemented with bFGF, VEGF, interleukin (IL)-6, IGF-1, IL-11, SCF, and EPO. Cells were maintained in a 5% CO₂, 5% O₂, and 95% humidity incubator. To harvest the CD34+ cells, the EBs were dissociated on day 8, cells were filtered through a 70 μm strainer, and CD34+ cells were isolated by CD34 magnetic bead staining.

### Results

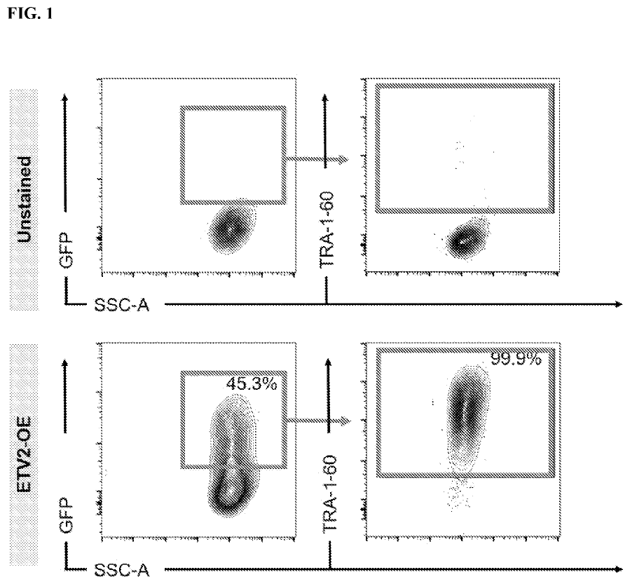

An adenoviral vector containing both ETV2 and GFP sequences under the control of the EF1A promoter was used to transduce induced pluripotent stem cells (iPSCs). After the transduction, about 45% of the iPSC culture was observed to be GFP positive, thus confirming ETV2 overexpression (ETV2-OE). It was further observed that ETV2-OE in iPSC cells preserves the pluripotency properties of iPSCs as shown by the sternness marker expression TRA-1-60 (FIG. 1). FIG. 1 shows FACS plots representative of transduction efficiency of iPSC with an adenoviral vector to overexpress the ETV2 and the GFP sequences.

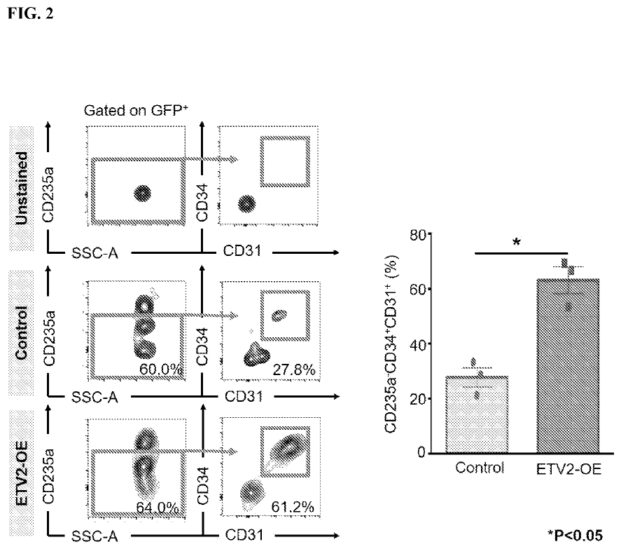

Next, the ETV2-OE-iPSCs were differentiated (along with control iPSCs transduced with a vector bearing the GFP sequence without ETV2) to embryoid bodies and subsequently to hemogenic endothelial cells (Strugeon et al., 2014). The results suggest that the overexpression of ETV2 boosts the formation of hemogenic endothelial cells as demonstrated by the expression of the CD34⁺ and CD31⁺markers within the CD235a⁻ population (FIG. 2). Specifically, FIG. 2 shows representative flow cytometric analysis of hemogenic endothelial cells (defined here as CD235a−CD34+CD31+) and relative quantification demonstrates that ETV2-OE enhances the formation of hemogenic endothelial cells as compared to controls.

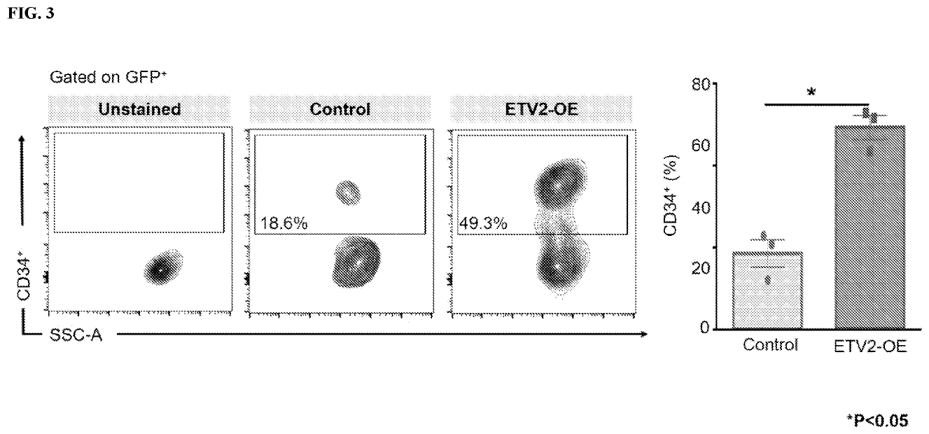

Moreover, the results suggest that ETV2-OE enhances the formation of the CD34⁺cells (FIG. 3). FIG. 3 shows representative flow cytometric analysis of CD34+ cells and relative quantification demonstrates that ETV2-OE enhances the CD34+ cell formation. Overall, these data indicate that ETV2 overexpression in iPSCs does not affect their pluripotency properties and facilitates their ability to undergo the hemogenic endothelial and hematopoietic differentiations.

### Example 2—iPSC-Derived HSCs Generated with Piezol Activation Undergo T Cell Differentiation Similar to Bone Marrow-Derived HSCs

### Methods

To analyze the EHT, EB-derived CD34+ cells were suspended in medium containing Y-27632, TPO, IL-3, SCF, IL-6, IL-11, IGF-1, VEGF, bFGF, BMP4, and FLT3. After the cells had adhered to the bottom of the wells for approximately 4-18 hours (by visual inspection), Yodal was added to the cultures for some experiments. After 4-7 days, the cells were collected for analysis.

iPSCs were differentiated to embryoid bodies for 8 days. At day 8, CD34+ cells from iPSC-derived embryoid bodies were harvested and cultured for additional 5 to 7 days to induce endothelial-to-hematopoietic (EHT) transition. Then, CD34+ cells were harvested from the EHT culture from day 5 to day 7 for further hematopoietic lineage differentiation.

CD34+ cells, harvested from the EHT culture between days 5-7 (or total of day 13-21 differentiation from iPSCs), were seeded in 48-well plates pre-coated with rhDL4 and RetroNectin. T lineage differentiation was induced in media containing aMEM, FBS, ITS-G, 2BME, ascorbic acid-2-phosphate, Glutamax, rhSCF, rhTPO, rhIL7, FLT3L, rhSDF-1a, and SB203580.

Between day 2 to day 6, 80% of the media was changed every other day. At D7, cells were transferred into new coated plates and analyzed for the presence of pro-T cells (CD34+CD7+CD5+/−).

Between day 8 to day 13, 80% of the media was changed every other day. At D14, 100,000 cells/wells were transferred to a new coated plate and the cells analyzed for the presence of pre-T cells (CD34−CD7+CD5+/−).

Between day 15 to day 20, 80% of the media was changed every other day. Cells were harvested at D21, and the cells were analyzed for CD3, CD8, CD5, CD7, TCRab expression, as surrogates for T cells, via FACS, and/or activated using CD3/CD28 beads to evaluate their functional properties.

After 21 days of differentiation, cells were collected and re-seeded at approximately 80,000 cells into new 96-well culture plates in RPMI 1640 (no L-glutamine; no phenol red) plus FBS, L-glutamine, IL-2, and then activated with 1:1 CD3/CD28 beads. After 72 hours of activation with CD3/CD28 beads, cells were analyzed for CD3, CD69, CD25 expression by FACS and IFN-γ expression using RT-qPCR. The supernatant was analyzed by ELISA.

### Results

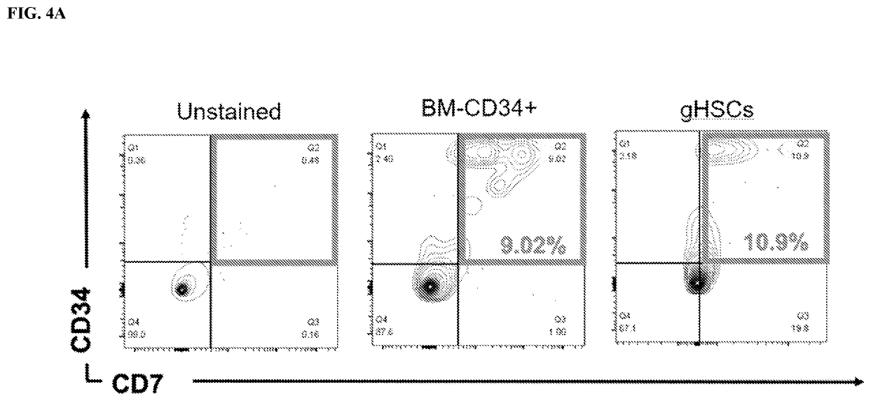

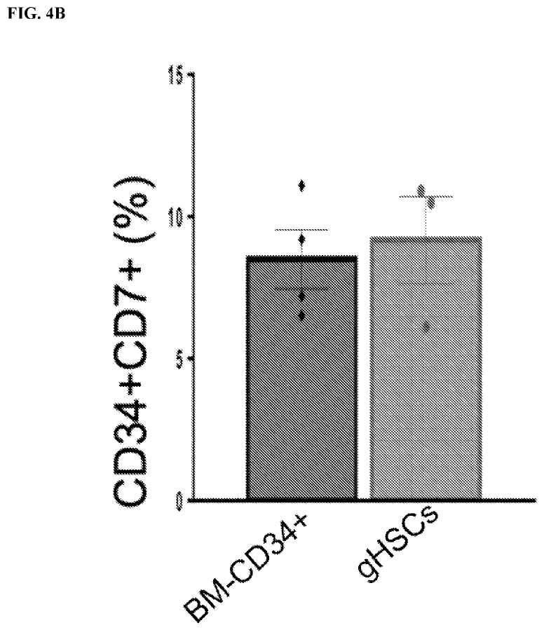

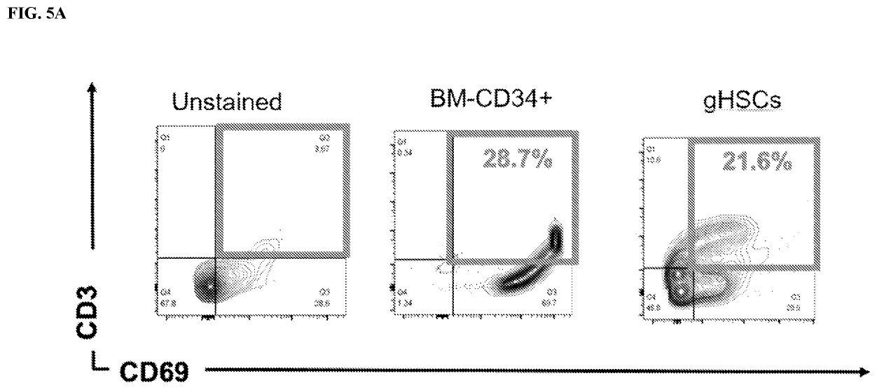

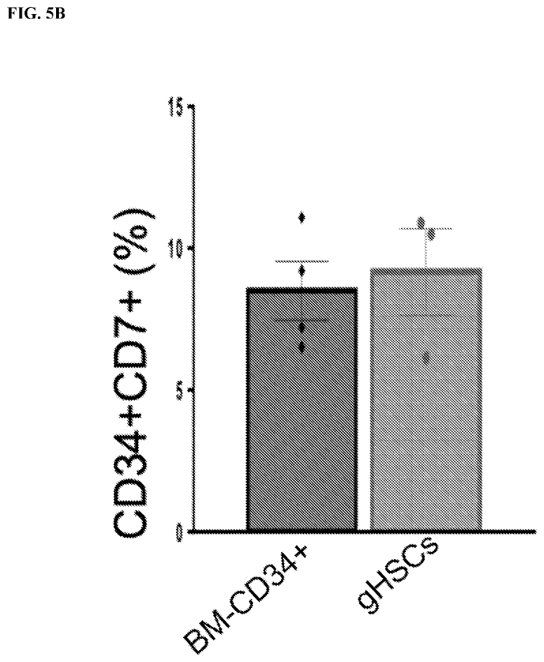

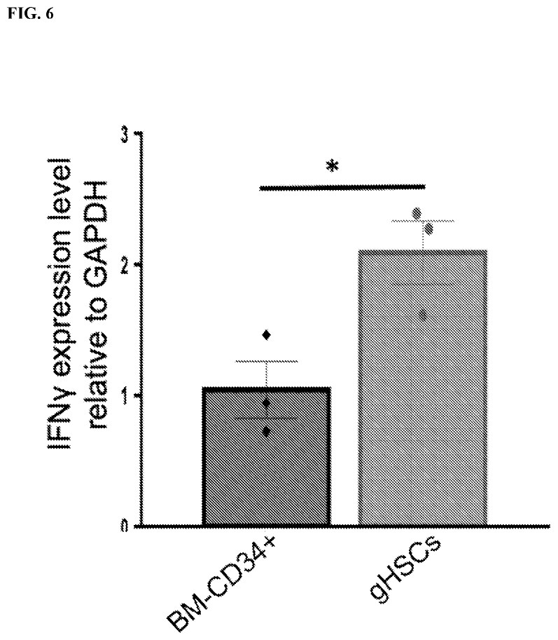

FIG. 4A and FIG. 4B show that iPSC-derived HSCs that are derived with EHT of CD34+ cells (CD34+ cells harvested at Day 8, and EHT was induced by Piezol activation) undergo pro-T cell differentiation similar to bone marrow (BM)-HSCs. Further, FIG. 5A and FIB. 5B show that iPSC-derived HSCs generated with EHT of CD34+ cells (with Piezol activation) undergo T cell differentiation and can be activated with CD3/CD28 beads similar to BM-HSCs. FIG. 6 shows that iPSC-derived HSCs generated with EHT of CD34+ cells (in this example with Piezol activation) can differentiate to functional T cells, as demonstrated by INFγ expression upon stimulation with CD3/CD28 beads. Together, these results demonstrate that EHT of CD34+ cells (optionally with Piezol activation) enhances the ability to further differentiate to hematopoietic lineages, such as progenitor T cells and functional T cells ex vivo.

iPSC-derived HSCs according to this disclosure, as well as lineages derived therefrom, can be a valuable therapy to reconstitute hematopoietic lineages, including cytotoxic T cells and natural killer cells, for oncological diseases. When used alongside therapies that target hematopoietic cells, such as for treatment of hematopoietic malignancies, elimination or reduced expression of targeted cancer antigens in the HSCs can provide for a powerful therapy.

### Example 3 CD33 Deletion

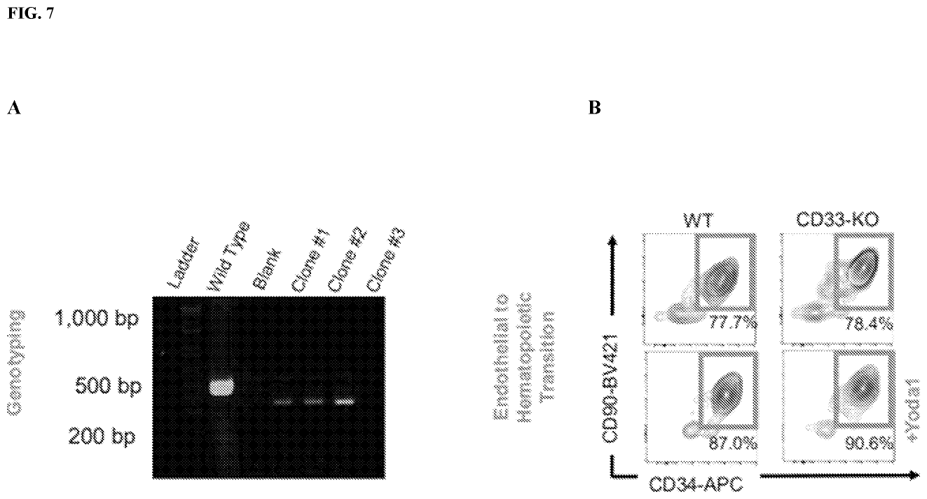

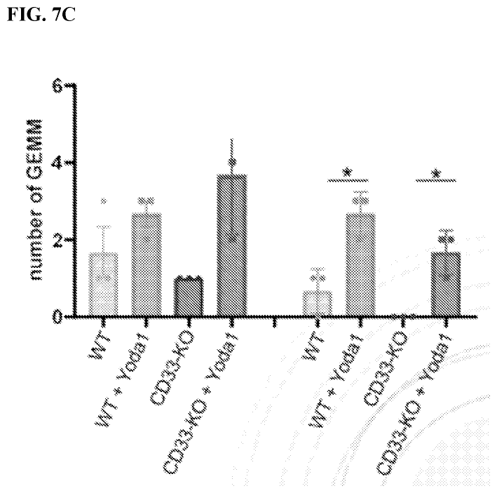

FIG. 7A shows generation of three CD33-KO iPSC clones. As shown in FIG. 7B, CD33-KO does not affect the ability of cells to undergo the endothelial-to hematopoietic transition. Further, CD33-KO does not affect the ability of cells to generate self-renewing HSCs (FIG. 7C). When used alongside therapies that target hematopoietic cells, such as for treatment of hematopoietic malignancies, elimination or reduced expression of CD33 in the HSCs (or cells derived therefrom) can provide for a powerful therapy.

### Example 4 CCR5 Deletion

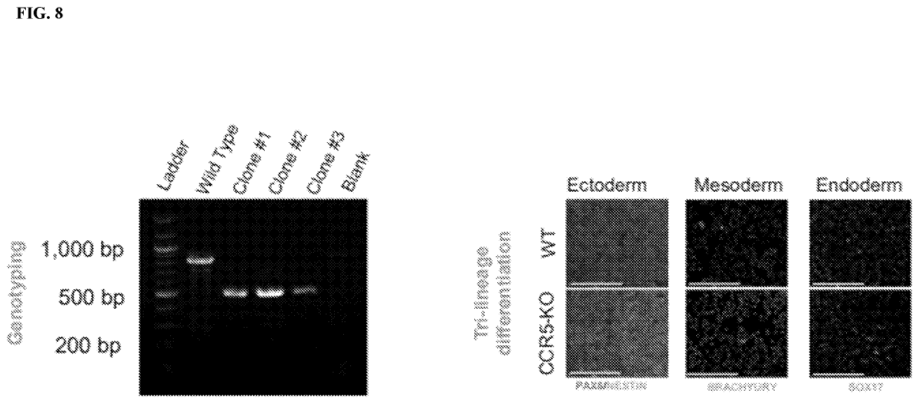

FIG. 8A shows generation of three CCR5-knockout (KO) iPSC clones. As shown in FIG. 8B, the CCR5-KO does not affect the iPSC pluripotency. Further, as shown in FIG. 8C, CCR5-KO does not affect the ability of cells to undergo the endothelial-to hematopoietic transition.

### Example 5 Evaluating Off-Target Editing in HLA Knockout HSCs

HLA typing of triple knockout (HLA edited) HSC clones was performed to check for unwanted editing and to ensure that no major editing events, e.g., deletion(s), occurred within other regions of chromosome 6. Sequencing methods and analyses were performed to evaluate the degree of gRNA off-target activity and to select gRNAs that represent a low risk of affecting non-target HLA genes.

Sequencing was performed by using in situ break labelling in fixed and permeabilized cells by ligating a full-length P5 sequencing adapter to end-prepared DSBs. Genomic DNA was extracted, fragmented, end-prepared, and ligated using a chemically modified half-functional P7 adapter. The resulting DNA libraries contained a mixture of functional DSB-labelled fragments (P5:P7) and non-functional genomic DNA fragments (P7:P7). Subsequent DNA sequencing of the DNA libraries enriched for DNA-labelled fragments, eliminating all extraneous, non-functional DNA. As the library preparation is PCR-free, each sequencing read obtained was equivalent to a single labelled DSB-end from a cell. This generated a DNA break readout, enabling the direct detection and quantification of genomic DSBs by sequencing without the need for error-correction and enabled mapping a clear list of off-target mutations.

Table 1 below summarizes the results of the editing strategy in two representative HILA edited clones relative to wild-type cells

TABLE 1
Clonal HSC HLA knockouts.

| Sample ID | Locus | Allele 1 | Allele 2 | Comments |
|---|---|---|---|---|
| WT (gHSC) | A | A*01:01:01 | A*01:01:01 | Not affected |
| | B | B*08:01:01 | B*08:01:01 | Not affected |
| | C | C*07:01:01 | C*07:01:01 | Not affected |
| | DPA1 | DPA1*01:03:01 | DPA1*02:01:02 | Not affected |
| | DPB1 | DPB1*01:01:01 | DPB1*04:01:01 | Not affected |
| | DQA1 | DQA1*05:01:01 | DQA1*05:01:01 | Not affected |
| | DQB1 | DQB1*02:01:01 | DQB1*02:01:01 | Not affected |
| | DRB1 | DRB1*03:01:01 | DRB1*03:01:01 | Not affected |
| | DRB345 | DRB3*01:01:02 | DRB3*01:01:02 | Not affected |
| Clone #1 | A | xxx | xxx | Deletion in Exon 2 |
| | B | B*08:01:01 | B*08:01:01 | Not affected |
| | C | C*07:01:01 | C*07:01:01 | Not affected |
| | DPA1 | DPA1*01:03:01 | DPA1*02:01:02 | Not affected |
| | DPB1 | xxx | xxx | Deletion in Exon 2 |
| | DQA1 | DQA1*05:01:01 | DQA1*05:01:01 | Not affected |
| | DQB1 | xxx | xxx | Delction in Exon 2 |
| | DRB1 | DRB1*03:01:01 | DRB1*03:01:01 | Not affected |
| | DRB345 | DRB3*01:01:02 | DRB3*01:01:02 | Not affected |
| Clone #2 | A | xxx | xxx | Deletion in Exon 2 |
| | B | B*08:01:01 | B*08:01:01 | Not affected |
| | C | C*07:01:01 | C*07:01:01 | Not affected |
| | DPA1 | DPA1*01:03:01 | DPA1*02:01:02 | Not affected |
| | DPB1 | xxx | xxx | Deletion in Exon 2 |
| | DQA1 | DQA1*05:01:01 | DQA1*05:01:01 | Not affected |
| | DQB1 | xxx | xxx | Deletion in Exon 2 |
| | DRB1 | DRB1*03:01:01 | DRB1*03:01:01 | Not affected |
| | DRB345 | DRB3*01:01:02 | DRB3*01:01:02 | Not affected |

Table 2 provides a non-limiting example of gRNAs used in the experiments which can be used to knock out expression of indicated HLA genes.

TABLE 2
Exemplary gRNA sequences

| gRNA ID | Spacer sequence |
|---|---|
| HLA-A | CGTAGCCCACGGCGATGAAG (SEQ ID NO: 1) |
| | ATTTCTTCACATCCGTGTCC (SEQ ID NO: 2) |
| | GTCTCCTGGTCCCAATACTC (SEQ ID NO: 3) |
| | GTAGCCCACGGCGATGAAGC (SEQ ID NO: 4) |
| | TAGCCCACGGCGATGAAGCG (SEQ ID NO: 5) |
| | GAGGGTTCGGGGCGCCATGA (SEQ ID NO: 6) |
| | CCGGAACACACGGAATGTGA (SEQ ID NO: 7) |
| | ACAGCGACGCCGCGAGCCAG (SEQ ID NO: 8) |
| | CCACTCACAGATTGACCGAG (SEQ ID NO: 9) |
| | CGACGCCGCGAGCCAGAGGA (SEQ ID NO: 10) |
| | CAGATTGACCGAGTGGACCT (SEQ ID NO: 11) |
| | CAGACTGACCGAGTGGACCT (SEQ ID NO: 12) |
| | ACAGACTGACCGAGTGGACC (SEQ ID NO: 13) |
| | GCCGGGCCGGGACACGGATG (SEQ ID NO: 14) |
| | CCAGGAGACACGGAATGTGA (SEQ ID NO: 15) |
| | CCAGTCACAGACTGACCGAG (SEQ ID NO: 16) |
| | TCGACAGCGACGCCGCGAGC (SEQ ID NO: 17) |
| HLA- | GAGATACATCTACAACCGGG (SEQ ID NO: 18) |
| DPB1 | TGGAGAGATACATCTACAAC (SEQ ID NO: 19) |
| | GGTTGTAGATGTATCTCTCC (SEQ ID NO: 20) |
| | GGAGAGATACATCTACAACC (SEQ ID NO: 21) |
| | AGGAATGCTACGCGTTTAAT (SEQ ID NO: 22) |
| HLA- | CAGATACATCTATAACCGAG (SEQ ID NO: 23) |
| DQB1 | CGAGTACTGGAACAGCCAGA (SEQ ID NO: 24) |
| | CCCGTTGGTGAAGTAGCACA (SEQ ID NO: 25) |
| | GTGCTACTTCACCAACGGGA (SEQ ID NO: 26) |
| | AGGTCGTGCGGAGCTCCAAC (SEQ ID NO: 27) |
| | AGGTGACAGTTGCTACCCGA (SEQ ID NO: 28) |
| | TCCCGTTGGTGAAGTAGCAC (SEQ ID NO: 29) |
| | AACTACGAGGTGGCGTACCG (SEQ ID NO: 30) |
| | CTGTTCCAGTACTCGGCAAC (SEQ ID NO: 31) |
| | ACTACGAGGTGGCGTACCGC (SEQ ID NO: 32) |
| | CGAAGCGCACGTACTCCTCT (SEQ ID NO: 33) |

The results show that the editing strategy was successful in selectively targeting the HLA-A, DPB1, and DQB1 genes without affecting the other HLA genes or introducing major deletion elsewhere.

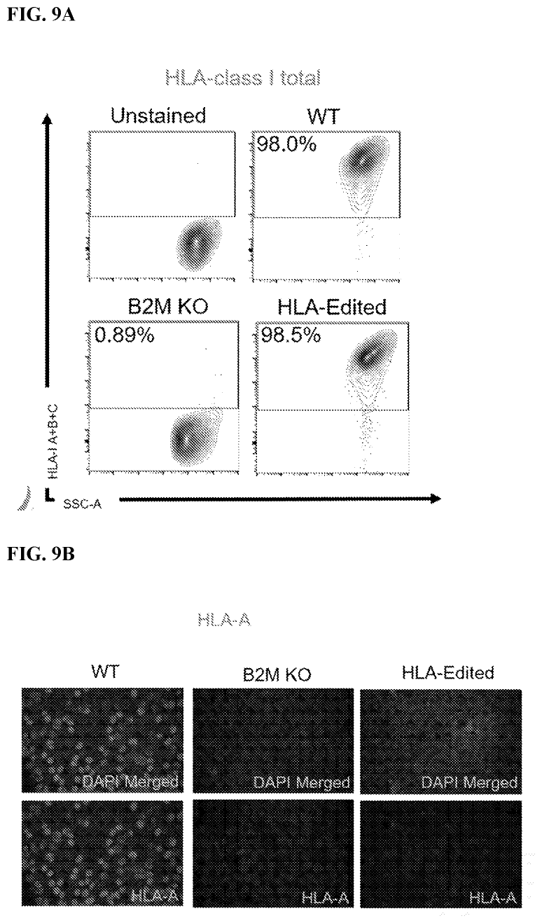

These results were confirmed by a phenotypic analysis of the HLA edited clones by FACS and immunofluorescence. As shown in FIGS. 9A and B, HLA edited cells tested positive for overall expression of HLA class-I molecules, comparable to the overall expression of HLA class-I molecules of wild-type cells. Specific expression of HLA-A via immunofluorescence confirmed that HLA-A was not expressed in the HLA edited cells, corroborating the finding that the gene editing strategy was successful in deleting only the HLA-A gene. Specifically, FIG. 9A, shows that the HLA edited cells were all positive for class-I like HLA to the same extent as the wild type cells (i.e., gHSC). This result indicates that despite the deletion of HLA-A, other class-I molecules like HLA-B and C were expressed and not affected by the gene editing strategy.

To confirm that the HLA-A gene was deleted, specific expression of the HLA-A was analyzed with immunofluorescence. As can be seen in FIG. 9B, HLA-A was not expressed in the HLA edited clone indicating that the gene editing strategy was efficient in specifically deleting the HLA-A gene only. Such preservation of overall class-I expression with deletion of HLA-A will facilitate patient matching while avoiding NK-cell mediated rejection.

### Example 6 Evaluating Pluripotency and Immunocompatibility of HLA Edited HSCs

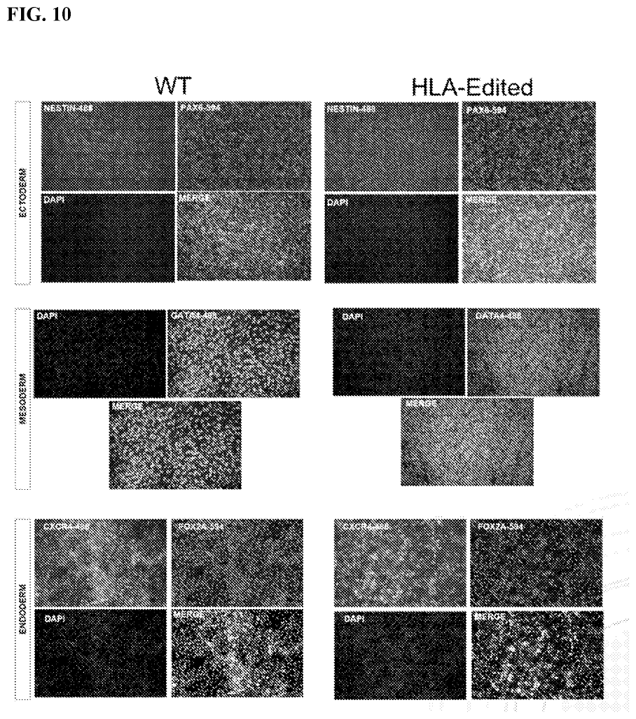

The ability of HLA edited cells to preserve pluripotency was evaluated. As shown in FIG. 10, immunofluorescence evaluation of the HLA edited iPSC clones indicated that they maintained trilineage differentiation, with ectoderm differentiation indicated by NESTIN-488 and PAX6-594 staining, mesoderm differentiation indicated by GATA-488 staining, and endoderm differentiation indicated by CXCR4-488 and FOX2A-594 staining.

HLA class I molecules are expressed on the surface of all nucleated cells and if the HLA class I molecules are mismatched between donor and recipient, then the cells could be recognized and killed by CD8+ T cells. Additionally, HLA mismatching could lead to cytokine release syndrome (CRS) and graft-versus-host disease (GVD). Conversely, the complete deletion of HLA-I molecules, via B2M KO, would make the cell a target of NK cell-mediated cytotoxicity. The preservation of overall class-I expression with deletion of HLA-A can facilitate patient matching while preventing the NK-cell mediated rejection. Thus, the immunocompatibility of the HLA edited HSCs was tested by co-culture with peripheral blood mononuclear cells (PBMCs) to evaluate if the immune cells would reject a graft of the HLA edited and wild type HSCs (gHSCs).

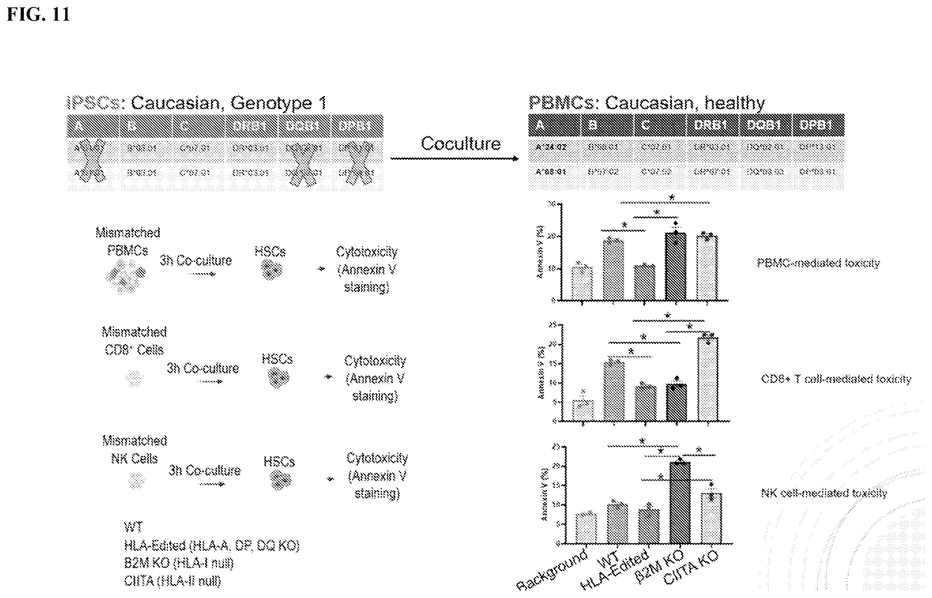

Wild-type (i.e., gHSCs) and HLA edited HSCs were co-cultured with PBMCs matching the HLA-B and HLA-C markers, but with mismatched HLA-A. B2M KO HSCs lacking expression of HLA class-I molecules and CIITA KO HSCs lacking expression of class-II molecules were used as controls to compare the degree of PBMC-mediated cytotoxicity for HLA-null and mismatched HLA, respectively. FIG. 11 shows the results of the PBMC-mediated cytotoxicity assay in the co-cultures as measured by an annexin V staining. The results show that deletion of HLA-A in the HLA edited HSCs protects the cells from PBMC-mediated cytotoxicity, while WT, B2M KO, and CIITA KO were susceptible to PBMC-mediated cytotoxicity. Co-cultured HSCs with sorted CD8+ T cells from the same PBMC donor protected HLA edited and B2M KO HSCs from CD8+ T cell cytotoxicity. Conversely, co-cultured HSCs with sorted NK cells only protected the WT and HLLA edited cells from the NK cell-mediated cytotoxicity.

In summary, the immune compatibility results show that the CD8+ T cells present in the PBMC samples were responsible for killing the cells with mismatched HLA molecules (WT) and CIITA KO, while the NK cells present in the PBMCs were responsible for killing the HLA-null cells (B2M KO). However, HLA edited HSCs are protected from CD8+ T cell-mediated cytotoxicity (because the mismatched HLA-A had been knocked out), and protected from NK cell-mediated cytotoxicity (because HLA class I molecule expression was largely preserved).

### Example 7—Evaluating the In Vivo Engraftment Potential of HLA Edited HSCs

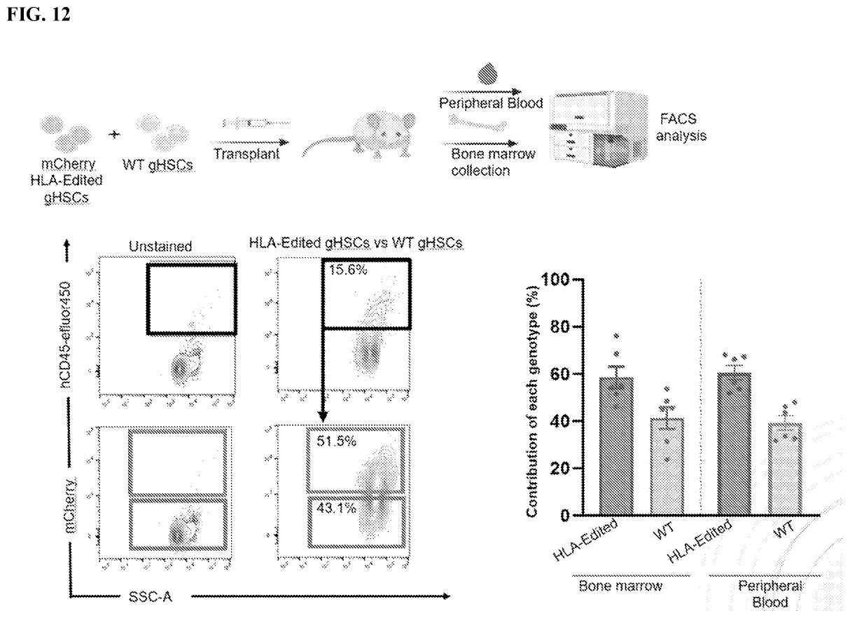

To evaluate the engrafting potential of HLA edited HSCs, the cells' ability to engraft in vivo was evaluated by a competitive transplant against WT HSCs. Equal proportions of mCherry HLA edited HSCs and wild-type HSCs (gHSCs) were admixed and transplanted into mice, from where bone marrow (BM) and peripheral blood samples were recovered and evaluated by FACS to compare the relative amounts of each cell type present in the samples. As shown in FIG. 12, both the HLA edited HSCs and the WT HSCs contributed to approximately equal engraftment in the BM and peripheral blood samples. These results confirm that HLA edited HSCs (prepared according to this disclosure) are comparable to WT HSCs in their engraftment and reconstitution potential. Hence, it is expected that properties of the WT HSCs are consistent with that of the HLA edited HSCs of the present disclosure, for generating T cell lineages.

### Example 8 Differentiation of HLA Edited HSCs to CD4+, CD8+ T Cells

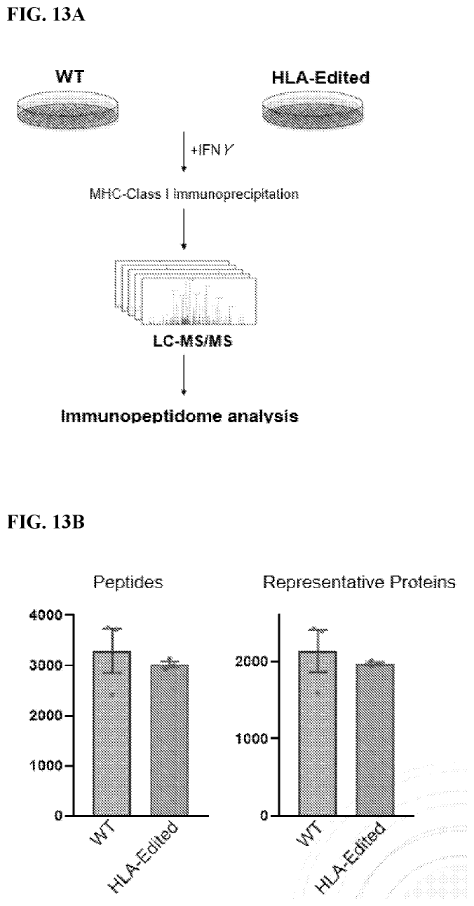

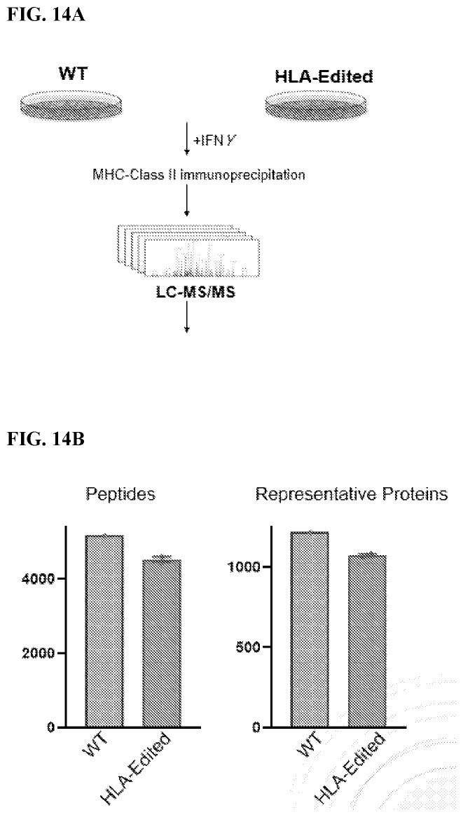

Antigen presenting cells (APCs) present antigens to helper CD4+ T cells through the HLA-II molecules. Activation of helper CD4+ T cells promotes the generation of antigen-specific CD8+ T cells which further develop into antigen-specific CTLs. Likewise, HLA Class I molecules are expressed on the surface of all nucleated cells and display peptide fragments of proteins from within the cell to CD8+ CTLs. CTLs induce cytotoxic killing of target (infected) cells upon recognition of HLA-I− peptide complex expressed on the cell surface. Hence, a study was carried out to determine if deletion of HLA-A impacts the edited HSCs' class I peptide presentation. As shown in FIGS. 13A and B, immunopeptidome analysis shows that the deletion of HLA-A does not impact overall class I peptide presentation. HLA-A edited cells showed comparable peptide and protein presentation when compared to wild type (i.e., unedited) HSCs. Further, as shown in FIGS. 14A and B, deletion of HLA-DQB1 and HLA-DPB1 does not impact overall class II peptide presentation by macrophages differentiated from the HSCs. Together, these data suggest that despite the deletion of HLA-A, HLA-DQ, and HLA-DP molecules, the cells (and their derived lineages) preserve their ability to present a broad spectrum of class I and II peptides.

### Example 7—In Vivo Testing of Antigen-Mediated Immune Response

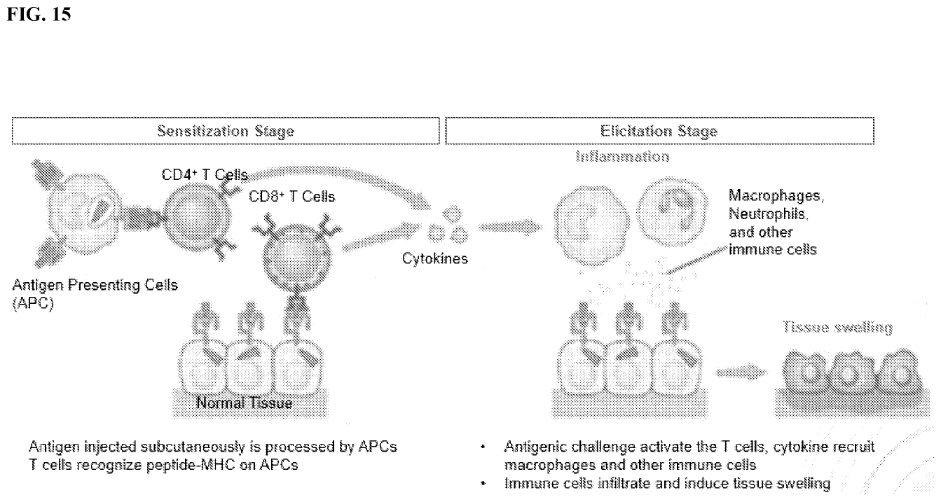

FIG. 15 is a schematic illustration of a Delayed Type Hypersensitivity Reaction, showing the sensitizing and eliciting stages of an antigen presentation. Briefly, upon antigen injection, antigen is processed by antigen presenting cells (APC) and presented by MHC Class II molecules on the APC surface. CD4+ T cells recognize peptide-MHC on antigen presenting cells (APCs). Upon antigenic challenge CD4+ helper T cells are activated and cytokines recruit macrophages and other immune cells, which induce tissue swelling.

A delayed-type hypersensitivity assay was performed on transplanted mice. Specifically, the mice were sensitized by subcutaneous injection of sheep Red blood cells as antigen. If the mice have a functional immune system, the APCs process the antigen and present peptide antigens to CD4+ T cells. Next, the mice were challenged by subcutaneous injection of the same antigen in the left paw. At this point the T cells are activated and secrete cytokines which recruit macrophages and other immune cells at the site of antigen injection creating tissue swelling. In this assay, a functional immune system resulted in the swelling of the left paw as measured with a micro caliper.

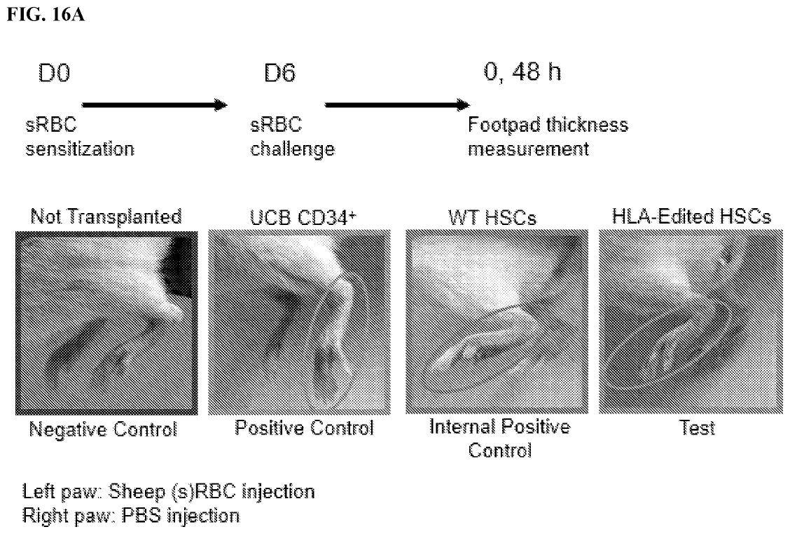

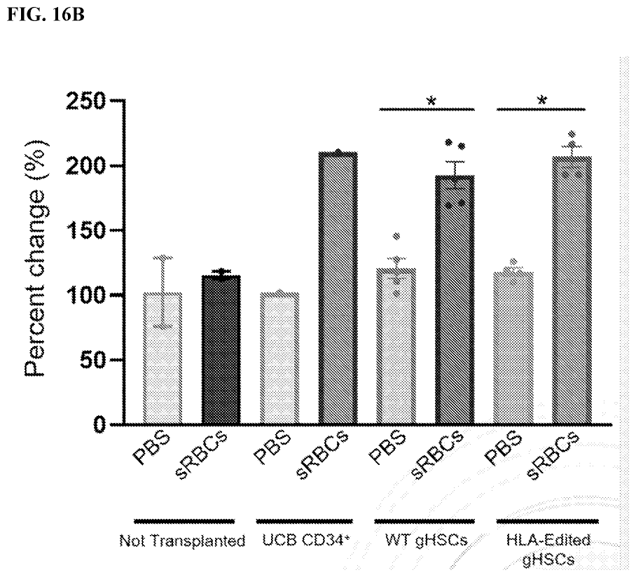

As can be seen in FIGS. 16A and B, the control (non-transplanted) mice did not show any left paw swelling as they are immunodeficient. Conversely, the mice transplanted with Cord Blood CD34+ cells showed tissue swelling and doubled the diameter of their left paw. A similar immune system response was found in both the mice transplanted with the WT (non-edited HSCs) and the HLA-edited HSCs (HLA edited).

### Example 9—Evaluating HSC-Derived T Cell (Pro-T Cell) Differentiation and Maturation

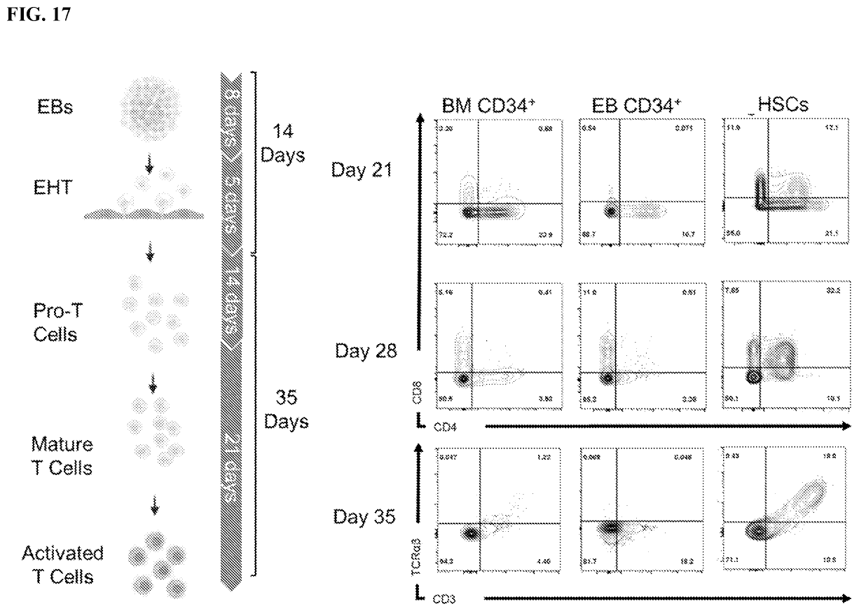

Next, the ability of the HSC-derived T cells (pro-T cells) to differentiate into mature T cells was tested. HSCs were generated via Peizol activation. After a 35-day differentiation period, pro-T cells were evaluated by cell sorting for the presence of CD4+, CD8+, and AB+T cell populations. As shown in FIG. 17, pro-T cells differentiated into CD4+, CD8+, and αβ+ T cells more efficiently than bone marrow (BM)-derived CD34+ cells and the CD34+ cells derived from the embryonic bodies (EB).

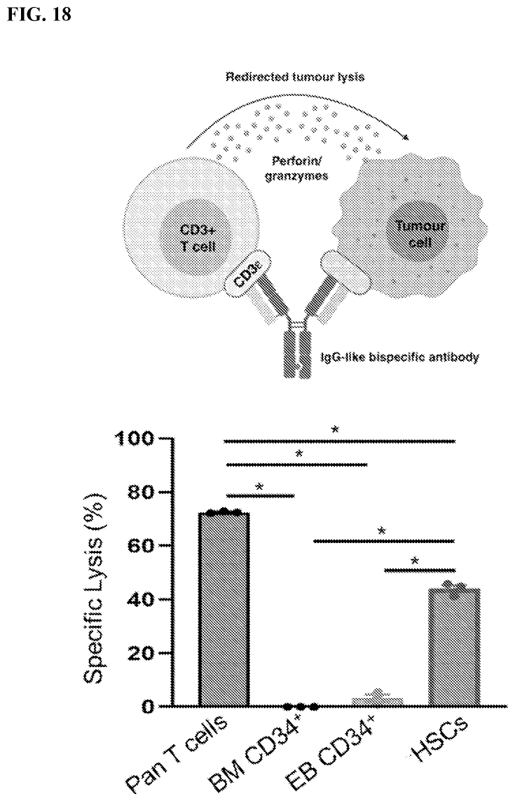

Next, to test the functional properties, each of the T cell populations were co-cultured with a CD19+ lymphoma cell line and an anti-CD3/CD-19 bispecific antibody. In this experimental model, the bispecific antibody engaged both the CD3 receptor on T cells and the CD19 cell surface receptor of the lymphoma cells, thus triggering T cell activation. The degree of activation was evaluated by measuring the subsequent T-cell mediated cytotoxicity in comparison to a Pan T cell control. As shown in FIG. 18, the pro-T cells exhibited a statistically significant outperformance in cytotoxicity in comparison to both the BM CD34+ T cells and the EB CD34+ T cells.

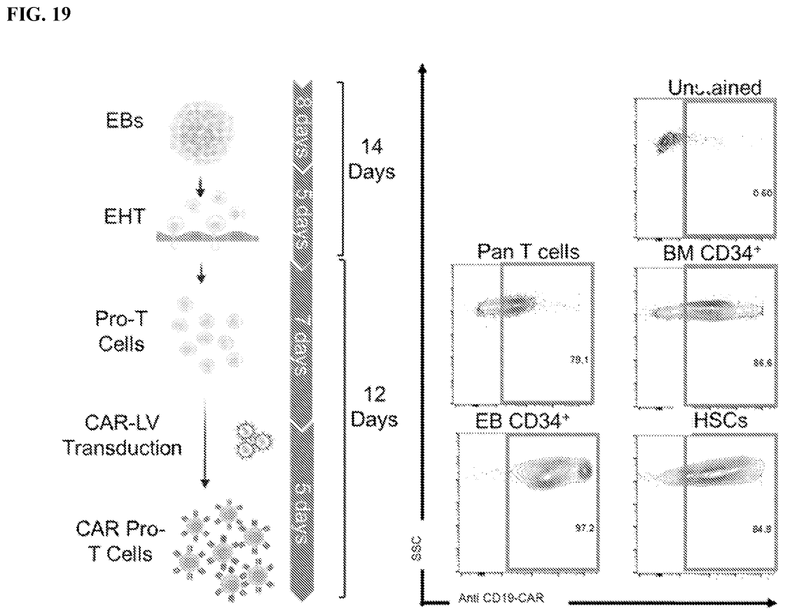

Because the overall differentiation process of pro-T cells is 35 days long, a transduction experiment was performed to test if the time required to differentiate the HSCs could be decreased. Pro-T cells were cultured in an activation media (for approx. 7 days) to increase the transduction efficiency of the cells. Next, the cells were transduced with lentiviral (LV) particles encoding an anti-CD19 CAR transgene. The cells were cultured for additional 4-5 days (a total of 12 days) and their maturation and killing capabilities were evaluated. As shown in FIG. 19, the HSC-derived pro-T cells can be transduced with high efficiency, with more than 80% of the cells express the anti-CD19 CAR as evidenced by cell sorting.

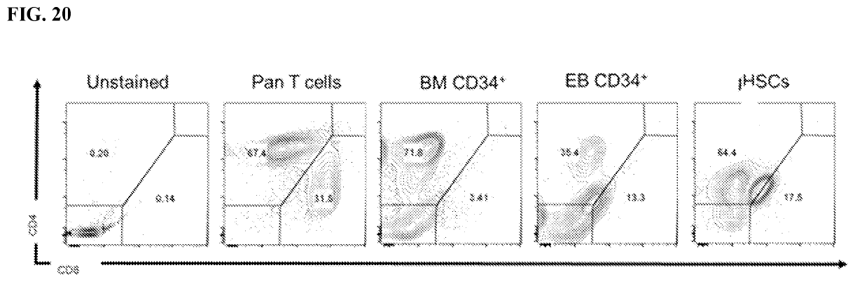

Next, the pro-T cells were evaluated for their ability to effectively mature into CD4+/CD8+ T cells via CAR transduction. The pro-T cells, along with bone marrow (BM)-derived CD34+ cells and CD34+ cells derived from the embryonic bodies (EB) (and Pan T cells as a positive control), underwent LV-transduction with the anti-CD19 CAR. The T cell subsets were screened by cell sorting for the presence of CD4 or CD8 cell surface marker expression. As shown in FIG. 20, the results indicated that CAR transduction promoted T cell maturation and that an increased degree of T cell maturation was observed in the pro-T cells in comparison to bone marrow (BM)-derived CD34+ cells and the CD34+ cells derived from the embryonic bodies (EB).

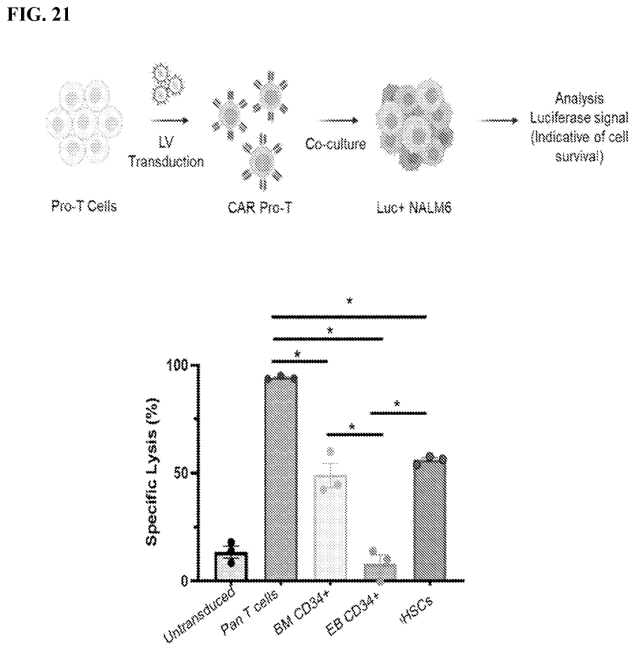

The ability of LV-transduced pro-T cells to function via anti-CD19 receptor-mediated cytotoxicity was evaluated. The T cell subsets were cocultured with a CD19+ leukemia cell line (NALM6) expressing a luciferase reporter gene (Luc+) to measure the degree of T cell-mediated cell lysis, with untransduced cells and Pan T cells as a negative and positive control, respectively. As shown in FIG. 21, the CAR pro-T cells effectively functioned via T cell-mediated lysis, demonstrating a degree of cytotoxicity comparable to the CAR-pro T cells derived from the BM CD34+ cells. Conversely, the CAR pro-T cells derived from the EB CD34+ cells showed no ability to kill the target cells.

### Example 10—Evaluating HSC Properties of Developing into Pro-T Cells

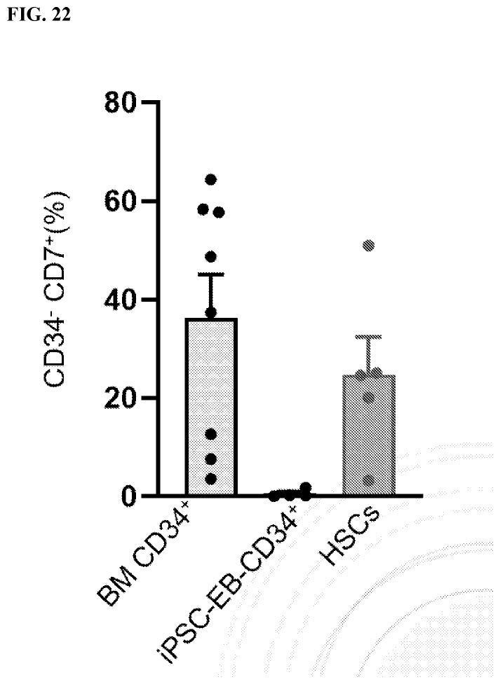

The ability of the HSCs to develop into pro-T cells was assessed by measuring the CD34-CD7+ markers on the pro-T cells. As shown in FIG. 22, FACS analysis showed that HSCs produced according to this disclosure successfully differentiated into CD34-CD7+ pro-T cells, as compared to bone marrow derived CD34+ cells or EB-derived CD34+ cells.

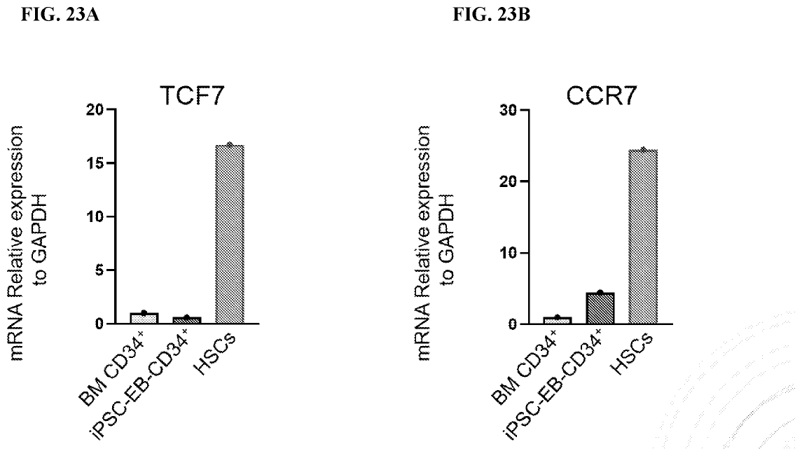

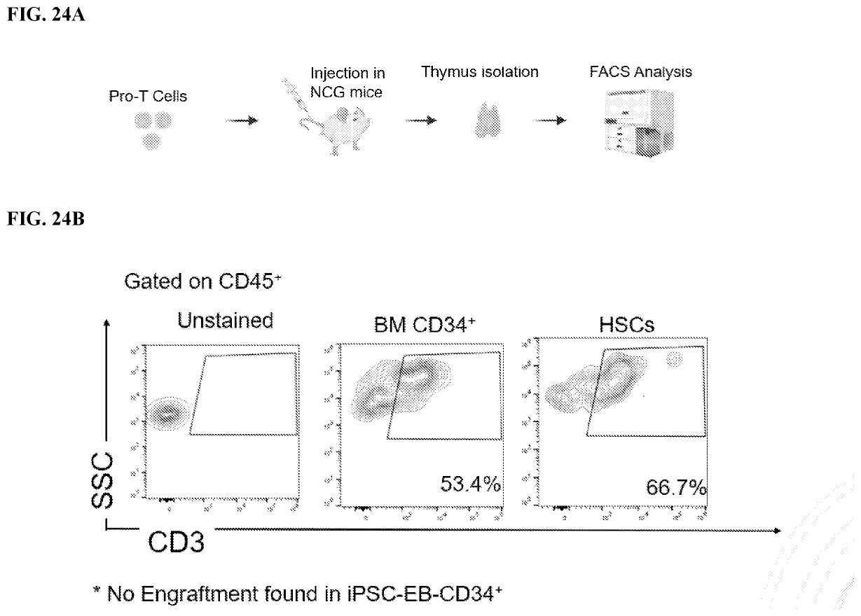

Next, the expression of T cell-specific transcription factors and thymus engrafting molecules were measured. FIG. 23A shows increased TCF7 expression and FIG. 23B shows increased CCR7 expression in the HSC-derived pro-T cells of the disclosure. FIG. 24A shows a schematic to determine whether HSC-derived Pro-T Cells engraft and differentiate in thymus. FIG. 24B shows FACS analysis of CD3 cell population of cells gated on a CD45+ cell population, which shows the superior engraftment and differentiation potential of the HSCs-derived Pro-T Cells in the thymus. Pro-T Cells of this example were prepared from HSCs using Piezol activation as already described.

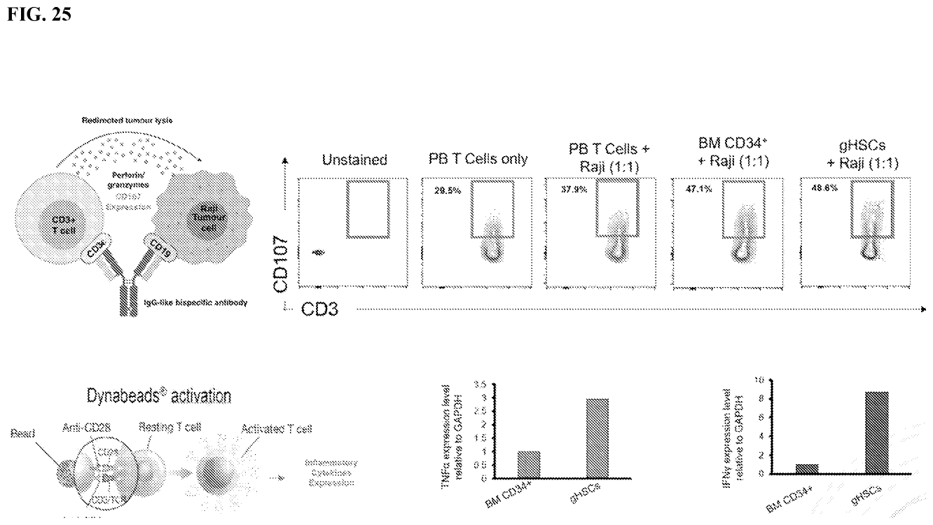

An in-vitro activation of the HSC-derived T cells were also measured, as illustrated in FIG. 25. Top panel of FIG. 25 shows FACS analysis of activated T cells from different sources, including the HSCs of the present disclosure (e.g., prepared using Piezol activation). T cells prepared from HSCs of the present disclosure demonstrated comparable or superior activation as measured by increased CD107 expression. The lower panel shows Dynabeads activation, where activated T cells express inflammatory cytokines. HSC-derived T cells according to the present disclosure (e.g., prepared using Piezol activation) expressed higher levels of inflammatory cytokines as exemplified by TNF-alpha and interferon gamma expression levels.

### Example 10—Evaluating Properties of CCR5 Knock Out HSCs to Develop into Pro-T Cells

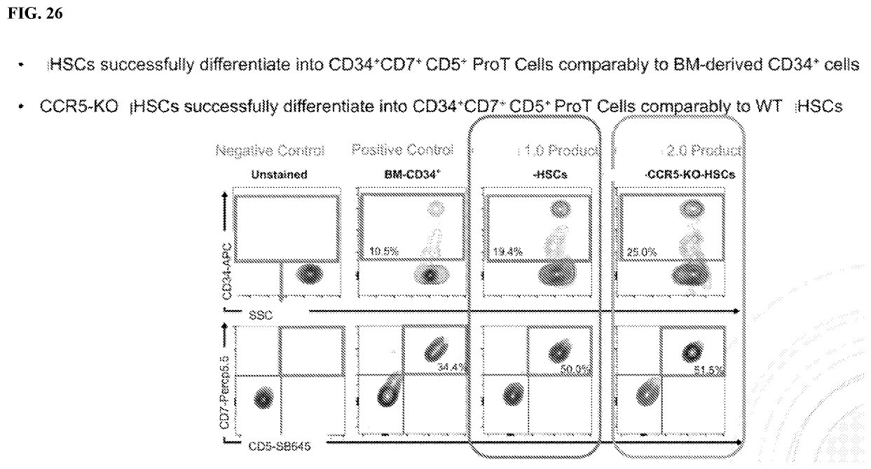

To determine if CCR5 knock out (CCR5-KO) HSCs can comparably differentiate to pro-T cells as their wild-type counterparts from which they are derived (i.e., HSCs of the present disclosure preparing via Piezol activation), a study was performed in which the CD34, CD7 and CD5 expression of the HSCs and the CCR5-KO were measured. As can be seen in FIG. 26, HSCs successfully differentiated into CD34+CD7+CD5+ pro-T cells comparably to bone marrow derived CD34+ cells. Likewise, the CCR5-KO, like their wild-type counterpart, successfully differentiated into CD34+CD7+CD5+ pro-T cells.

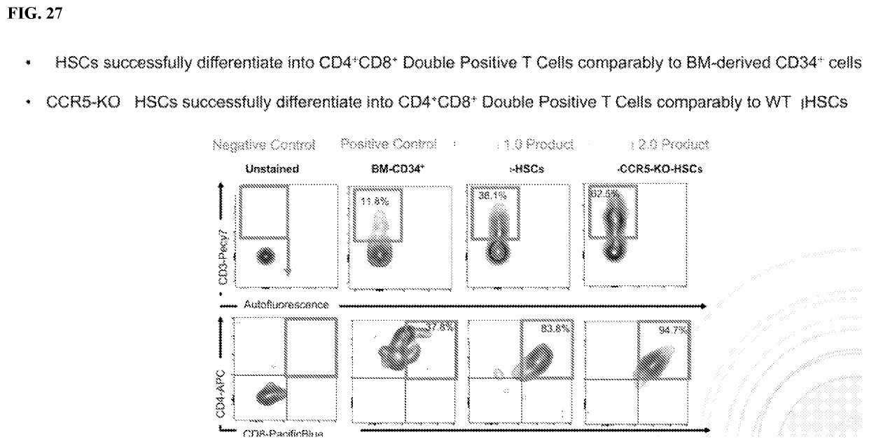

Next, the property of CCR5-knocked out HSCs to differentiate into double positive (CD4+CD8+) T cells was assessed and was it comparable to the HSCs from which they were derived. As can be seen in FIG. 27, CCR5-knocked out HSCs comparably differentiated into double positive (CD4+CD8+) T cells when compared to their wild type counterparts from which they were derived (i.e., HSCs of the present disclosure prepared via Piezol activation).

1. A method for preparing a population of hematopoietic stem cells (HSCs) and/or hematopoietic stem progenitor cells (HSPCs), the method comprising:
preparing a human induced pluripotent stem cell (iPSC) population;
modifying one or more endogenous genes in the iPSC population that are tumor-associated antigens, thereby disrupting the expression of the tumor associated antigen;
inducing differentiation of the iPSC population to a CD34+ population; and
inducing endothelial-to-hematopoietic transition (EHT) of the CD34+ population for at least 2 days and no more than 12 days, to prepare a population comprising HSCs and/or HSPCs having reduced expression of the tumor-associated antigen.

2. The method of claim 1, wherein the tumor associated antigen is expressed or overexpressed in one or more of cytogenetically normal acute myeloid leukemia (CN-AML), acute myeloid leukemia (AML), acute lymphoid leukemia, chronic myelogenous leukemia, acute lymphoblastic leukemia, chronic lymphoblastic leukemia, chronic lymphoid leukemia, Hodgkin's lymphoma, non-Hodgkin's lymphoma, and multiple myeloma.

3. The method of claim 2, wherein the endogenous gene is expressed in immune cells.

4-9. (canceled)

10. The method of claim 1, wherein at least one of the endogenous genes is selected from CD33, CD19, CD7, CD123, and CD371.

11-25. (canceled)

26. The method of claim 1, wherein the iPSC population is derived from lymphocytes, cord blood cells, peripheral blood mononuclear cells, CD34+ cells, or human primary tissues.

27. (canceled)

28. The method of claim 26, wherein the iPSCs are homozygous for one or more HLA Class I and/or Class II genes.

29. The method of claim 28, wherein the iPSCs are homozygous for HLA-DRB1.

30. (canceled)

31. The method of claim 28, wherein the iPSCs are gene-edited to delete one or more HLA Class I genes, delete one or more Class II genes, and/or delete one or more genes governing HLA or MHC expression or presentation capacity.

32-34. (canceled)

35. The method of claim 31, wherein the iPSCs are: HLA-A^{neg}, homozygous for both HLA-B and HLA-C, HLA-DPB1^{neg}, HLA-DQB1^{neg}, and homozygous for HLA-DRB1.

36. The method of claim 1, wherein the disruption of the one or more endogenous genes is generated by introducing (a) a Cas9 endonuclease or a nucleic acid encoding a Cas9 endonuclease, and (b) a nucleic acid molecule encoding a guide RNA (gRNA) directing the mutation or deletion of nucleotide sequences of the endogenous gene by the Cas9 endonuclease.

37-39. (canceled)

40. The method of claim 36, wherein the endothelial-to-hematopoietic transition (EHT) generates an HSC population comprising one or more of long-term hematopoietic stem cells (LT-HSCs), short-term hematopoietic stem cells, and hematopoietic stem progenitor cells.

41-45. (canceled)

46. The method of claim 1, wherein the induction of endothelial-to-hematopoietic transition (EHT) comprises Piezol activation; wherein the Piezol activation is by contacting the iPSCs or cells derived from the iPSCs, with one or more Piezol agonists.

47-51. (canceled)

52. An HSC population that is:
(i) derived from iPSCs by EHT of CD34+ cells dissociated from embryoid bodies;
(ii) has a genetically-disrupted expression of one or more endogenous genes that are tumor-associated antigens;
(iii) proliferates in vivo but does not exhibit uncontrolled growth or tumor formation in vivo; and
(iv) differentiates in vivo to reconstitute hematopoietic lineages.

53. The HSC population of claim 52, wherein the iPSCs are homozygous for one or more HLA Class I and/or Class II genes.

54-55. (canceled)

56. The HSC population of claim 52, wherein the iPSCs are gene-edited to delete one or more HLA Class I genes, delete one or more Class II genes, and/or delete one or more genes governing HLA or MHC expression or presentation capacity.

57-60. (canceled)

61. An HSC population that has a genetically-disrupted expression of one or more endogenous genes that are tumor-associated antigens; wherein the iPSCs are: HLA-A^{neg}, homozygous for both HLA-B and HLA-C, HLA-DPB1^{neg}, HLA-DQB1^{neg}, and homozygous for HLA-DRB1.

62. The HSC population of claim 61, wherein the tumor-associated antigen is expressed or overexpressed in one or more of cytogenetically normal acute myeloid leukemia (CN-AML), acute myeloid leukemia (AML), acute lymphoid leukemia, chronic myelogenous leukemia, acute lymphoblastic leukemia, chronic lymphoblastic leukemia, chronic lymphoid leukemia, Hodgkin's lymphoma, non-Hodgkin's lymphoma, and multiple myeloma.

63. The HSC population of claim 62, wherein the endogenous gene is expressed in immune cells.

64-72. (canceled)

73. A method for treating a subject having a cancer and undergoing a treatment that targets cancer cells expressing a tumor associated antigen, comprising administering to the subject a therapeutically effective dose of the HSC populations of claim 52, wherein the HSCs have a reduced expression of the tumor associated antigen.

74. The method of claim 73, wherein the treatment targeting cells expressing a tumor associated antigen is a T cell therapy, a CAR-T therapy, a CAR-NK cell therapy, an antibody-based therapy, or an antibody drug conjugate.

75-79. (canceled)
